(19) United States
(12) Patent Application Publication
(10) Pub. No.: US 2023/0047393 A1
(43) Pub. Date: Feb. 16, 2023

(54) COMPOSITION CONTAINING AN ALK5 INHIBITOR, EW-7197

(71) Applicant: EWHA DrugDesignHouse Co.,Ltd., Seoul (KR)

(72) Inventors: Noriaki Nishida, Osaka (JP); Dae-Kee Kim, Seoul (KR)

(73) Assignee: EWHA DrugDesignHouse Co.,Ltd., Seoul (KR)

(21) Appl. No.: 17/391,202

(22) Filed: Aug. 2, 2021

(51) Int. Cl. A61K 31/444; A61K 47/10; A61K 47/34

(52) U.S. Cl. CPC A61K 31/444; A61K 47/10; A61K 47/34

(57) ABSTRACT

Disclosed is a pharmaceutical composition containing EW-7197 (2-fluoro-N-((5-(6-methylpyridin-2-yl)-4-([1,2,4]triazolo[1,5-a]pyridin-6-yl)-1H-imidazol-2-yl)methyl)aniline), a pharmaceutically acceptable salt thereof, or a solvate thereof as an active ingredient useful in the treatment and prevention of a disease state mediated by transforming growth factor-β (TGF-β) type I receptor (ALK5).

### BACKGROUND

### (a) Technical Field

The present invention relates to a pharmaceutical composition containing EW-7197 as an active ingredient useful in the treatment and prevention of a disease state mediated by transforming growth factor-β (TFG-β) type I receptor (ALK5).

### (b) Background Art

There are three distinct homodimeric mammalian TGF-β isoforms designated as TGF-β1, TFG-β2, and TFG-β3, which are pleiotropic modulators of cell proliferation and differentiation, wound healing, extracellular matrix (ECM) production, and immunosuppression. The term “TFG-β” refers to a composition comprising one or more of those distinct TGF-βs. All TGF-βs are synthesized as 390 to 412 amino acid precursors that undergo proteolytic cleavage to produce the mature forms, which consist of the C-terminal 112 amino acids. In their mature, biologically active forms, TFG-βs are acid- and heat-stable disulfide-linked homodimers of two polypeptide chains of 112 amino acids each. Comparison of the amino acid sequence of human TFG-β1, TFG-β2, and TFG-β3 has demonstrated that the three proteins exhibit about 70-80% sequence identity in their mature forms.

TFG-β transduces signals through two highly conserved single transmembrane serine/threonine kinases, the type I (ALK5) and type II TFG-β receptors. Upon ligand induced oligomerization, the type II receptor hyperphosphorylates serine/threonine residues in the GS region of the ALK5, which leads to activation of the ALK5 by creating a binding site for Smad proteins. The activated ALK5 in turn phosphorylates Smad2 and Smad3 proteins at the C-terminal SSXS-motif thereby causing their dissociation from the receptor and heteromeric complex formation with Smad4. Smad complexes, then, translocate to the nucleus, assemble with specific DNA-binding co-factors and co-modulators to finally activate transcription of ECM components and inhibitors of matrix-degrading proteases.

U.S. Pat. No. 8,080,568 B1 discloses 2-pyridyl substituted imidazoles as ALK5 and/or ALK4 inhibitors, and U.S. Pat. No. 8,513,222 B2 discloses their use for treating fibrosis, cancer, and vascular injuries. Especially, one of the representative compounds claimed in U.S. Pat. Nos. 8,080,568B1 and 8,513,222 B2, EW-7197 (2-fluoro-N-((5-(6-methylpyridin-2-yl)-4-([1,2,4]triazolo[1,5-a]pyridin-6-yl)-1H-imidazol-2-yl)methyl)aniline), with the following structure:

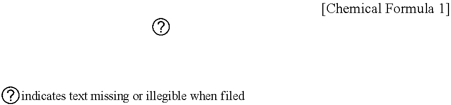

[symbol]

demonstrates its high efficacy in various animal models. EW-7197 (TEW-7197; vactosertib) decreases the expression of collagen, a-smooth muscle action (aSMA), fibronectin, 4-hydroxy-2,3-nonenal, and integrins in the liver of CCl₄-treated mice and bile duct ligated rats, in the lung of bleomycin-treated mice, and in the kidney of mice with unilateral ureteral obstruction (e.g., Park, S.-A. et al., Cell. Mol. Life Sci. 72: 2023-2039 (2015)).

Self-expanding metallic stent (SEMS) placement is a well-established method for treating malignant esophageal strictures, however, this procedure has not gained widespread acceptance for treating benign esophageal strictures because of granulation tissue formation. EW-7197 suppresses granulation tissue formation after SEMS placement in the rat esophagus and possesses strong potential as an anti-fibrotic agent via its ability to inhibit TGF-β signaling (e.g., Jun, E. J. et al., Gastrointest. Endosc. 86(1): 219-228 (2017)). An EW-7197-eluting nanofiber-covered stent suppresses granulation tissue formation after stent placement in a canine urethral model, showing that mean thicknesses of the papillary projection, thickness of submucosal fibrosis, number of epithelial layers, and degree of collagen deposition are significantly lower in the drug stent group than in the control stent group (all p< 0.001) (e.g., Han, K. et al., PLoS ONE 13(2): e0192430 (2018)).

Postsurgical peritoneal adhesions are pathologic bonds (a thin film of connective tissue or a thick fibrous band containing blood vessels and nerves) between abdominal organs and the peritoneum. Adhesion formation is a normal response to peritoneal injury, occurring in approximately 95% of patients after laparotomy. Although this process is integral to the healing of the peritoneum, adhesions can occasionally cause significant morbidity, including small-bowel obstruction and female infertility, as well as chronic abdominal or pelvic pain, or both (e.g., ten Broek, R. P. et al., BMJ (Clin. Res. Ed.) 347: f5588 (2013)). EW-7197 reduces the incidence, quality, and tenacity of peritoneal adhesions in a dose-dependent manner by inhibiting TGF-β1/Smad2/3-induced mesothelial-to-mesenchymal transition in a rat model (e.g., Tsauo, J. et al., Surgery 164: 1100-1108 (2018)) and prevents the frequency and the stability of adhesion bands in a mouse model (e.g., Soleimani, A. et al., J Cell. Physiol. 235: 1349-1357 (2020)).

Inflammatory bowel disease (IBD) is defined as chronic intestinal inflammation and includes ulcerative colitis and Crohn's disease. TGF-β is involved in the maintenance of intestinal homeostasis through modulating the functions of immune cells, the epithelium, and the luminal microbiota, which are associated with the pathogenesis of IBD (e.g., Ihara, S. et al., J. Gastroenterol. 52: 777-787 (2017)). In a mouse model of ulcerative colitis, EW-7197 ameliorates the clinical symptoms of colitis, suppresses overexpression of proinflammatory and profibrotic genes, and inhibits excessive collagen deposition and fibrosis in colitis tissues (e.g., Binabaj, M. M. et al., J. Cell. Physiol. 234: 11654-11661 (2019)).

TGF-β signaling in the tumor microenvironment significantly impacts carcinoma initiation, progression, and metastasis via epithelial cell autonomous and interdependent stromal-epithelial interactions in vivo (e.g., Stover, D. G J. Cell. Biochem. 101: 851-861 (2007)). ALK5 inhibitors, EW-7197 and LY-2157299, suppress the progression of melanoma with enhanced cytotoxic T-lymphocyte (CTL) responses in a mouse B16 melanoma model, and orally administered EW-7197 (2.5 mg/kg, once daily) is more effective than LY-2157299 (75 mg/kg, bid) (e.g., Yoon, J.-H. et al., EMBO Mol. Med. 5: 1720-1739 (2013)). EW-7197 inhibits Smad/TGF-β signaling, cell migration, invasion, and lung metastasis in MMTV/c-Neu mice and 4T1 orthotopic-grafted mice and enhanced CTL activity in 4T1 orthotopic-grafted mice, indicating its potential as an anticancer therapeutic (Son, J. Y. et al., Mol. Cancer Ther. 13(7): 1704-1716 (2014)). Blocking TGF-β signaling with EW-7197 suppresses paclitaxel-induced epithelial-mesenchymal transition (EMT) and cancer stem-like cells properties in a MDA-MB-231-xenografted mouse model (e.g., Park, S.-Y. et al., Oncotarget 6(35): 37526-37543 (2015)). Combined treatment with EW-7197 and a tyrosine kinase inhibitor, ponatinib, significantly delays disease relapse and prolongs survival in CML-affected mice, compared to the ponatinib treatment alone (e.g., Naka, K. et al., Cancer Sci. 107(2): 140-148 (2016)).

A number of ocular diseases are associated with increased TFG-β signaling in the eye.

Glaucoma is the second leading cause of irreversible blindness in the world, and the most common form of glaucoma is primary open-angle glaucoma (POAG). In POAG, excessive ECM deposition within the trabecular meshwork results in increased resistance to outflow of aqueous humour causing intraocular pressure (IOP) and consequent damage to retinal neurons leading to neurodegeneration and irreversible blindness. In 2013, the number of people (aged 40-80 years) with POAG worldwide was estimated to be 44.1 million, increasing to 52.7 million in 2020 and 79.8 million in 2040 (e.g., Tham, Y-C. et al., Ophthalmology 121: 2081-2090 (2014)). TFG-β2 levels are increased in nearly half of the eyes with primary POAG and in most of the eyes with juvenile glaucoma in the aqueous humor of eyes (e.g., Picht, G et al., Graefes Arch. Clin. Exp. Ophthalmol. 239: 199-207 (2001)). Both TFG-β1 and TFG-β2 isoforms are reported to increase ECM production in cultured human Tenon's capsule fibroblasts derived from patients with pseudoexfoliation glaucoma and POAG (e.g., Kottler, U. B. et al., Exp. Eye Res. 80: 121-134 (2005)). Increased amounts of TFG-β2 is found in aqueous humor and reactive optic nerve astrocytes in patients with POAG (Wang, J. et al., J. Glaucoma 26: 390-395 (2017)). TFG-β2 is reported to be a key player contributing to the structural changes in the ECM of the trabecular meshwork and optic nerve head as characteristically seen in POAG (e.g., Fuchshofer, R. and Tamm, E. R., Cell Tissue Res. 347: 279-290 (2012)).

Glaucoma filtration surgery (GFS) is commonly performed when medication fails to control IOP adequately. However, post-surgical scarring after GFS remains a critical determinant of the long-term surgical outcome and IOP after drainage surgery. The antimetabolites, mitomycin C and 5-fluorouracil, are the current gold standards used primarily to prevent fibrosis after GFS, but lead to non-specific cytotoxicity and potentially blinding complications such as hypotony maculopathy and infection (e.g., Yu-Wai-Man, C. and Khaw, P. T. Expert Rev. Ophthalmol. 10(1): 65-76 (2015)). US 2011/0160210 A1 discloses treatment of glaucoma and control of intraocular pressure using ALK5 modulating agents. One of the representative compounds claimed in US 2011/0160210 A1, an ALK5 inhibitor, SB-431542, reduces the level of fibronectin in TFG-β2-treated perfused human anterior segments and the levels of fibronectin and plasminogen activator inhibitor-1 (PAI-1) in TFG-β2-treated trabecular meshwork cell cultures. SB-431542 decreases post-surgical scarring and fibrosis after GFS in a rabbit model (e.g., Xiao, Y Q. et al., Invest. Ophthalmol. Vis. Sci. 50(4): 1698-1706 (2009)). Another ALK5 inhibitor, SB-505124, inhibited TFG-β activity and promoted bleb survival in a rabbit model of trabeculectomy (e.g., Sapitro, J. et al., Mol. Vis. 16: 1880-1892 (2010)). WO 2010/121162 A1 discloses the use of TFG-β receptor inhibitors to suppress ocular scarring. One of the representative compounds claimed in US 2010/121162 A1, SB-505124, prevents ocular scarring following GFS in a rabbit model.

Cataract is a progressive clouding of the normally clear lens that may cause partial or total blindness, which is one of the most prevalent eye diseases and accounts for much of the world's blindness. Cataract is caused by abnormalities in differentiation into fibroblasts of lens epithelium cells, abnormal proliferation of lens epithelium cells, and the loss of transparency of the lens due to the pathological buildup of extracellular substrates (e.g., Kim, D. H. et al., J. Korean Ophthalmol. Soc. 46(8): 1393-1400 (2005)). These cataract changes in the lens are attributed to changes in cytokine network, mainly related to TFG-β signaling, that regulate cell proliferation and differentiation (e.g., Lovicu, F. J. et al., Exp. Eye Res. 142: 92-101 (2016)). When TFG-β is added to the rat lens epithelial cell culture, pathological changes in cataracts such as formation of spindle-shaped cells, capsule wrinkling, cell death by apoptosis, and accumulation of ECM are observed (e.g., Hales, A. M. et al., Invest. Ophthalmol. Vis. Sci. 35(2): 388-401 (1994); de Iongh, R. U. et al., Exp. Eye Res. 72(6): 649-659 (2001)). In addition, two molecular markers for subcapsular cataract, αSMA and type I collagen, are observed in the lens or artificial lens treated with TGF-β, indicating that TFG-β is important factor in the development of cataracts (e.g., Hales, A. M. et al., J. Exp. Med. 185(2): 273-280 (1997); Symonds, J. G et al., Exp. Eye Res. 82(4): 693-699 (2006)).

Currently, the most commonly used treatment for cataract is surgical removal of the lens cells and subsequent implantation of a synthetic replacement lens within the remaining lens capsule. However, following the mechanical insult of surgery, the remaining lens epithelial cells rapidly grow and could ultimately encroach on the visual axis where light scattering changes induced by the cells can give rise to secondary visual loss, which is known as posterior capsule opacification (PCO; secondary cataract) (e.g., Wormstone, I. M. et al., Exp. Eye Res. 88(2): 257-269 (2009)), occurring in 20% to 40% of patients 2 to 5 years after surgery. TGF-β-induced trans-differentiation of lens epithelial cells into myofibroblastic/fibroblastic cells appears to play a key role in this process (e.g., Symonds, J. G et al., Exp. Eye Res. 82(4): 693-699 (2006)). An EMT is central to fibrotic PCO and forms of fibrotic cataract, and TGF-β has been shown to induce lens EMT (e.g., Lovicu, F. J. et al., Exp. Eye Res. 142: 92-101 (2016)). Currently, none of the pharmacological therapies using either anti-inflammatory agents (e.g., dexamethasone) or antimetabolites (e.g., mitomycin C, 5-fluorouracil) is effective and safe enough for the prevention of PCO. Pirfenidone, a non-selective ALK5 inhibitor, inhibits TGF-β-induced proliferation, migration, and EMT of human lens epithelial cells line SRA01/04 at nontoxic concentrations (e.g., Yang, Y. et al., PLoS ONE 8(2): e56837 (2013)).

Wet age-related macular degeneration (AMD) with the hallmark presence of choroidal neovascularization (CNV) is one of the main causes of blindness in the world. With developing wet AMD, the patient's central vision is distorted and becomes progressively deficient, eventually resulting in blindness. Angiogenesis on the choroidal membrane is the major characteristic of wet AMD (e.g., Wang, K. et al., Acta Biochim. Biophys. Sin. 51(1): 1-8 (2019)). These neovascular blood vessels cause hemorrhage, leading to the formation of a disciform scar with rapid visual impairment (e.g., Kliffen, M. et al., Br. J. Ophthalmol. 81(2): 154-162 (1997)). Intravitreal injection of anti-VEGF antibody such as bevacizumab, ranibizumab, and aflibercept is currently considered as the gold standard therapy for wet AMD, however, over 60% of wet AMD patients do not have improved vision after the treatment (e.g., Lu, H. et al., PLoS ONE 9(1): e87530 (2014)). Therefore, development of new therapeutic approaches or combination therapies with anti-VEGF antibody is needed. Emerging evidence has shown that TFG-β signaling plays a significant role in the progression of wet AMD, indicating that blocking of TFG-β signaling is a potential target for wet AMD treatment. TFG-β is highly expressed in the retinal pigment epithelium (RPE) in patients with wet AMD and in a CNV mouse model, further confirming the importance of TFG-β in wet AMD (e.g., Bai, Y et al., Mol. Vis. 20: 1258-1270 (2014)). TFG-β significantly stimulates angiogenesis by inducing the production of other pro-angiogenic factors like VEGF in RPE cells and enhances vascular permeability (e.g., Kliffen, M. et al., Br. J. Ophthalmol. 81(2): 154-162 (1997)). Treatment of human RPE cells with low dose of TFG-β2 for 24 and 48 h in vitro increases secretion of VEGF by 5- and 9-folds, respectively (e.g., Bian, Z.-M., et al., Exp. Eye Res. 84(5): 812-822 (2007)). Actually, a neutralizing antibody against TFG-β strongly inhibits angiogenesis in vitro and in vivo (e.g., Goumans, M.-J., et al., Cell Res. 19: 116-127 (2009)). In addition to stimulating angiogenesis, TFG-β recruits inflammatory cells, which, in turn, initiate other proangiogenic cytokine-release cascades. In the later phase of CNV, fibroblasts become more proliferative, leading to scar tissue formation, in which collagen remodeling and scar contraction are partially mediated by TFG-β (e.g., Bai, Y. et al., Mol. Vis. 20: 1258-1270 (2014)). In subretinal fibrosis mice, treatment with a TFG-β antibody remarkably reduces the degree of induced subretinal fibrosis (e.g., Zhang, H. et al., Int. J. Ophthalmol. 5(3): 307-311 (2012)). An ALK5 inhibitor, LY-2157299, significantly (p<0.05, n=5 per group) reduces the neovascular area in retina of mice at day 14 afer laser-induced CNV formation when compared with that of PBS treated control groups (e.g., Wang, X. et al., Sci. Rep. 7: 9672 (2017)).

Proliferative vitreoretinopathy (PVR) is a common cause for treatment failure after rhegmatogenous retinal detachment surgery. PVR is characterized by EMT of RPE cells and consecutive formation of fibrous membranes, leading to retinal redetachment. In PVR-induced pigmented rabbits, development of PVR membranes is accompanied by a pronounced upregulation of TGF-β1 (e.g., Hoerster, R. et al., Graefes Arch. Clin. Exp. Ophthalmol. 252: 11-16 (2014)). In a rabbit PVR trauma model, intravitreal injection of an ALK5 inhibitor, LY-364947, prevents PVR development significantly and subsequent tractional retinal detachment (e.g., Nassar, K. et al., Exp. Eye Res. 123: 72-86 (2014)).

Proliferative diabetic retinopathy (PDR) is a serious ocular complication of diabetes and is characterized by retinal neovascularization and microvascular leakage in response to chronic ischemia. VEGF and TFG-β cooperate to induce both retinal neovascularization and fibrosis around these new vessels, which may potentially cause retinal detachment or bleeding (e.g., Saika, S. Lab. Invest. 86: 106-115 (2006)). Although anti-VEGF therapy alongside pan-retinal photocoagulation has been shown to reduce neovascularization and macular edema (e.g., Gulkilik, G et al., Int. Ophthalmol. 30: 697-702 (2010)), response to anti-VEGF treatment is heterogeneous (e.g., Elman, M. J. et al., Ophthalmology 122: 375-381 (2015)). Therefore, treatment with an anti-VEGF antibody in combination with an ALK5 inhibitor may increase therapeutic efficacy in PDR patients.

Fuchs' endothelial corneal dystrophy (FECD) is a slowly progressive bilateral disease of corneal endothelium in which accumulation of ECM and loss of corneal endothelial cells are phenotypic features. The only therapy for corneal haziness due to corneal endothelial diseases, including FECD, is corneal transplantation using donor corneas, and no pharmaceutical treatment is available. The expression levels of TFG-β isoforms and TFG-β receptors are high in the corneal endothelium of patients with FECD, suggesting that inhibition of TFG-β signaling could be a novel therapeutic target that suppresses cell loss as well as the accumulation of ECM in FECD (e.g., Okumura, N. et al., Sci. Rep. 7(1): 6801 (2017)). EP 3725313A1 discloses composition or method including EW-7197 for treating or preventing corneal endothelial diseases. In a FECD animal model, Col8a2 knock-in mice, EW-7197 (0.02% eye drop administration) sufficiently migrates into the corneal endothelium and effectively suppresses a decrease in corneal endothelial cells and/or corneal endothelial disorders represented by overexpression of ECM (type I collagen, fibronectin, and the like). In the same animal model above, EW-7197 suppresses fibronectin expression at a broad range of concentrations (0.004%, 0.02%, and 0.1%) in a dose-dependent manner.

Congenital ectopia lentis (CEL) is a displacement or malposition of the eye's crystalline lens from its normal location. A significant correlation exists between high levels of aqueous homor TFG-β2 and the severity of ectopia lentis in patients with CEL. Aqueous humor TGF-β2 levels in the CEL patients are significantly higher compared with those in congenital cataract patients (e.g., Cao, Q. et al., Mol. Med. Rep. 20: 559-566 (2019)).

Hypertrophic scar is a common disease after tissue injury, especially after burn injury. Clinically, it results in tissue hypertrophy and severe contracture, leading to functional disability and facial organ disfigurement. Pathologically, it is characterized with overproduction and deposition of ECM, cell overgrowth and irregular distribution, enhanced angiogenesis, and enhanced transformation of fibroblasts to myofibroblast. Overexpressions of TFG-β and its receptors have been discovered in hypertrophic scar and keloid tissues as well as their derived fibroblasts when compared with normal skin and normal fibroblasts. A TFG-β antagonist effectively reduces hypertrophic scar formation in a porcine model (e.g., Singer, A. J. et al., J. Burn Care Res. 30: 329-334 (2009)).

An anastomotic stricture is a type of pathological scar healing process of surgical incisions in the blood vessel, bowel, esophagus, urinary tract, etc. After surgical repair of the esophagus, the levels of TGF-β1 protein and mRNA in the tissues collected from the patients with stenosis are significantly up-regulated as compared with those from the control group (e.g., Zhao, H. et al., Arch. Med. Sci. 11(4): 770-778 (2015)).

In order to use EW-7197 for treating or preventing TFG-β signaling related ocular diseases, postsurgical peritoneal adhesions, postsurgical anastomotic strictures, hypertrophic scar, or keloid in human, formulation development of an aqueous solution containing appropriate amounts of EW-7197 is needed. However, EW-7197 has a very low water solubility of approximately 10 to 20 μg/mL in an acceptable pH range for ophthalmic solution, as shown in Table 1, it is difficult to prepare an aqueous solution containing appropriate amounts of EW-7197.

TABLE 1
Solubility of EW-7197 at various pH values

| | | Solubility (mg/ml) | | | |
|---|---|---|---|---|---|
| | | | | Average | pH after |
| pH | Buffer | Trial 1 | Trial 2 | (n = 2) | equilibration |
| 1.2 | Hydrochloric acid | 25.06 | 25.04 | 25.05 | 2.81 |
| | buffer | | | | |
| 2.0 | Hydrochloric acid | 2.93 | 2.88 | 2.905 | 2.97 |
| | buffer | | | | |
| 3.0 | Citrate buffer | 1.00 | 1.03 | 1.015 | 3.30 |
| 4.0 | Citrate buffer | 0.11 | 0.1 | 0.105 | 4.16 |
| 5.0 | Citrate buffer | 0.019 | 0.021 | 0.020 | 4.90 |
| 6.0 | Phosphate buffer | 0.012 | 0.012 | 0.012 | 5.96 |
| 6.8 | Phosphate buffer | 0.011 | 0.013 | 0.012 | 6.79 |
| 7.4 | Phosphate buffer | 0.013 | 0.013 | 0.013 | 7.32 |
| — | Water | 0.009 | 0.009 | 0.009 | 7.17 |
| 12.86 | 0.1N NaOH | 0.396 | 0.478 | 0.437 | 12.53 |

The inventors developed a technique of preparing a composition containing EW-7197 at about 0.001% w/v to about 0.5% w/v.

### SUMMARY OF THE DISCLOSURE

The present invention has been completed by developing a polyethylene glycol (PEG)/poloxamer formulation containing EW-7197 from about 0.1% w/v to about 0.5% w/v. The present invention provides, for example, the following items.

(Item 1)

A composition for treating or preventing a disease state mediated by transforming growth factor-β (TFG-β) type I receptor (ALK5) in human, comprising an effective amount of EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof.

(Item 2)

The composition of item 1, wherein the composition is for reducing the accumulation of excess extracellular matrix (ECM) in human by inhibiting the TFG-β signaling pathway, for example, inhibiting the phosphorylation of Smad2 or Smad3 by ALK5.

(Item 3)

The composition of item 1 or 2, wherein a disease state mediated by ALK5 or accumulating excess ECM by inhibiting the TFG-β signaling pathway is glaucoma, glaucoma filtration surgery bleb failure, intraocular pressure, cataract, posterior capsule opacification (PCO; secondary cataract), corneal haze, wet age-related macular degeneration (AMD), proliferative vitreoretinopathy (PVR), proliferative diabetic retinopathy (PDR), Fuchs' endothelial corneal dystrophy (FECD), congenital ectopia lentis (CEL), postsurgical peritoneal adhesions, postsurgical anastomotic strictures, hypertrophic scar, or keloid.

(Item 4)

The composition of any one of items 1 to 3, wherein EW-7197 or a pharmaceutically acceptable salt thereof, or a solvate thereof is in the composition at a concentration of about 0.001% w/v to about 0.5% w/v.

(Item 5)

The composition of any one of items 1 to 4, wherein EW-7197 or a pharmaceutically acceptable salt thereof, or a solvate thereof is in the composition at a concentration of about 0.01% w/v to about 0.2% w/v.

(Item 6)

The composition of any one of items 1 to 5, wherein said composition further comprises an ophthalmologically acceptable solvent, diluting agent, liquid vehicle, preservative, stabilizer, solubilizer, viscosity enhancer, penetration enhancer, tonicity agent, gelling agent, buffering agent, wetting agent, or antioxidant.

(Item 7)

The composition of item 6, wherein said solubilizer is tyloxapol, polysorbate 80, PEG-40 stearate (MYS-40), PEG-60 hydrogenated castor oil (HCO-60), poloxamer, polyethylene glycol (PEG), PEG/tyloxapol, or PEG/poloxamer.

(Item 8)

The composition of item 6 or 7, wherein preferred solubilizer is PEG/poloxamer.

(Item 9)

The composition of item 7 or 8, wherein preferred poloxamer is poloxamer 188 or poloxamer 407, and preferred PEG is PEG4000.

(Item 10)

The composition of any one of items 1 to 9, which is an eye drop.

### BRIEF DESCRIPTION OF THE DRAWINGS

The above and other features of the present invention will now be described in detail with reference to certain exemplary embodiments thereof, illustrated in the accompanying drawings, which are given hereinbelow by way of illustration only, and thus are not limitative of the present invention, and wherein:

FIG. 1a to 1d shows thermal stability of EW-7197 in buffers (25° C.),

FIG. 2a to 2b shows photostability of EW-7197 in buffers (ultraviolet and visible light),

FIG. 3 shows solubility of EW-7197 with various solubilizing agents (4° C. and 25° C.),

FIG. 4a to 4b shows solubility of EW-7197 at various concentrations of surfactant, PEG4000, and niacinamide (4° C. and 25° C.),

FIG. 5a to 5b shows solubility of EW-7197 when tyloxapol, PEG4000, and niacinamide are used in combination (4° C. and 25° C.),

FIG. 6a to 6b shows thermal stability of EW-7197 with various surfactants (40° C.),

FIG. 7a to 7c shows thermal stability of EW-7197 with various stabilizing agents (40° C.),

FIG. 8 shows HPLC chromatograms of the formulation containing no stabilizing agent and the formulation containing EDTA,

FIG. 9a to 9b shows thermal stability of formulations with sodium thiosulfate (25° C. and 40° C.),

FIG. 10 shows thermal stability of EW-7197 in various buffers (40° C.),

FIG. 11a to 11b shows effect of metal ion on thermal stability of EW-7197 (40° C.),

FIG. 12a to 12b shows solubility of EW-7197 in PEG/poloxamer formulation (5° C. and 25° C.),

FIG. 13 shows thermal stability of EW-7197 at various concentrations of poloxamer 407 (40° C.) (formulation number: N05, N06, and N07),

FIG. 14 shows thermal stability at various concentrations of EW-7197 (40° C.) (formulation number: N04, N08, N12, and N13), and

FIG. 15 shows thermal stability of 25% PEG4000/10% poloxamer 407 formulation of EW-7197 with various viscous agents (40° C.).

### DETAILED DESCRIPTION

The objects described above, and other objects, features and advantages of the present invention, will be clearly understood from the following preferred embodiments with reference to the attached drawings. However, the present invention is not limited to the embodiments, and may be embodied in different forms. The embodiments are suggested only to offer a thorough and complete understanding of the disclosed context and to sufficiently inform those skilled in the art of the technical concept of the present invention.

Unless the context clearly indicates otherwise, all numbers, figures and/or expressions that represent ingredients, reaction conditions, polymer compositions and amounts of mixtures used in the specification are approximations that reflect various uncertainties of measurement occurring inherently in obtaining these figures, among other things. For this reason, it should be understood that, in all cases, the term “about” should be understood to modify all numbers, figures and/or expressions. In addition, when numerical ranges are disclosed in the description, these ranges are continuous and include all numbers from the minimum to the maximum including the maximum within each range unless otherwise defined. Furthermore, when the range refers to an integer, it includes all integers from the minimum to the maximum including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include numbers such as 10%, 11%, 12% and 13% as well as 30%, and any sub-ranges of 10% to 15%, 12% to 18%, or 20% to 30%, as well as any numbers, such as 10.5%, 15.5% and 25.5%, between appropriate integers that fall within the range.

The present invention is described hereinafter. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of a contradiction, the present specification (including the definitions) takes precedence.

As used herein, “pharmaceutically acceptable salt” refers to an inorganic or organic acid addition salt of the compound of the invention that is relatively non-toxic. These salts can be prepared by reacting a compound purified temporarily between the final isolation and purification of a compound or by a free base form separately with a suitable organic or inorganic salt, and isolating a salt formed in this manner.

Examples of pharmaceutically acceptable basic salts of the compound of the invention include alkali metal salts such as sodium salts and potassium salts; alkaline earth metal salts such as calcium salts and magnesium salts; ammonium salts; aliphatic amine salts such as trimethylamine salts, triethylamine salts, dicyclohexylamine salts, ethanolamine salts, diethanolamine salts, triethanolamine salts, procaine salts, meglumine salts, diethanolamine salts, and ethylenediamine salts; aralkylamine salts such as N,N-dibenzylethylenediamine and benetamine salts; heterocyclic aromatic amine salts such as pyridine salts, picoline salts, quinoline salts, and isoquinoline salts; quaternary ammonium salts such as tetramethylammonium salts, tetraethylammonium salt, benzyltrimethylammonium salts, benzyltriethylammonium salts, benzyltributylammonium salts, methyltrioctylammonium salts, and tetrabutylammonium salts; basic amino acid salts such as arginine salts and lysine salts; and the like.

Examples of pharmaceutically acceptable acidic salts of the compound of the invention include inorganic acid salts such as hydrochlorides, sulfates, nitrates, phosphates, carbonates, hydrogen carbonates, and perchlorates; organic acid salts such as acetates, propionates, lactates, maleates, fumarates, tartrates, malates, citrates, and ascorbates; sulfonates such as methanesulfonates, isethionates, benzenesulfonates, and p-toluenesulfonates; acidic amino acids such as aspartates and glutamates; and the like.

As used herein, “solvate” refers to a solvate of the compound of the invention or a pharmaceutically acceptable salt thereof, encompassing, for example, a solvate of an organic solvent (e.g., alcohol (ethanol or the like)-ate), hydrate, and the like. When forming a hydrate, this can be coordinated with any number of water molecules. Examples of hydrates include monohydrates, dihydrates, and the like.

The preferred embodiments are described hereinafter. It is understood that the embodiments are exemplification of the present invention, so that the scope of the present invention is not limited to such preferred embodiments. It should be understood that those skilled in the art can refer to the following preferred embodiments to readily make modifications or changes within the scope of the present invention. Any of these embodiments can be appropriately combined by those skilled in the art.

In one aspect, the present invention provides a composition for treating or preventing a disease state mediated by ALK5, comprising an effective amount of EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof.

In some embodiments, the composition of the invention is for reducing the accumulation of excess ECM by inhibiting the TFG-β signaling pathway, for example, inhibiting the phosphorylation of Smad2 or Smad3 by ALK5.

In another aspect, the present invention provides a composition for treating or preventing glaucoma, glaucoma filtration surgery bleb failure, intraocular pressure, cataract, posterior capsule opacification (PCO; secondary cataract), corneal haze, wet age-related macular degeneration (AMD), proliferative vitreoretinopathy (PVR), proliferative diabetic retinopathy (PDR), Fuchs' endothelial corneal dystrophy (FECD), congenital ectopia lentis (CEL), postsurgical peritoneal adhesions, postsurgical anastomotic strictures, hypertrophic scar, or keloid, comprising an effective amount of EW-7197 or a pharmaceutically acceptable salt thereof, or a solvate thereof.

The composition of the invention can be a pharmaceutical composition (e.g., eye drop, intracameral injection, intravitreal injection, subconjunctival injection, or intraperitoneal administration for postsurgical peritoneal adhesions and strictures). A pharmaceutical composition can further comprise a pharmaceutically acceptable carrier. Examples of pharmaceutically acceptable carriers include, but are not limited to, any solvents, diluting agents, liquid vehicles, preservatives (e.g., benzoic acid, benzalkonium chloride, benzethonium chloride, benzyl alcohol, butylparaben, cetrimonium bromide, cetylpyridinium chloride, chlorobutanol, chlorocresol, cresol, ethylparaben, methylparaben, methylparaben sodium, phenol, phenoxyethanol, phenylethyl alcohol, phenylmercuric acetate, phenylmercuric nitrate, potassium benzoate, potassium sorbate, propylparaben, propylparaben sodium, sodium metabisulfite, sodium benzoate, sodium dehydroacetate, sodium propionate, sorbic acid, thimerosal, and thymol), stabilizers (e.g., disodium edetate dihydrate (EDTA), sodium thiosulfate hydrate, dibutylhydroxytoluene (BHT), mannitol, polyethylene glycol 4000 (PEG4000), niacinamide, and hypromellose 60SH-4000 (HPMC 60SH-4000)), solubilizers (e.g., poloxamer 188, poloxamer 338, poloxamer 407, polysorbate 20, polysorbate 80, tyloxapol, PEG-40 stearate (MYS-40), PEG-60 hydrogenated castor oil (HCO-60), benzalkonium chloride, metolose SM-25 (MC SM-25), hypromellose 60SH-50 (HPMC 60SH-50), hypromellose 60SH-4000 (HPMC 60SH-4000), carmellose sodium (CMC), povidone K-30 (PVP), lipidure-PMB (Lipidure), polyethylene glycol 400 (PEG400), polyethylene glycol 4000 (PEG4000), propylene glycol (PG), glycerin, and niacinamide), viscosity enhancers (e.g., xantan gum, carmellose sodium (CMC), and hypromellose 60SH-4000 (HPMC 60SH-4000)), penetration enhancers (e.g., benzalkonium chloride, polyoxyethylene glycol ethers (lauryl, stearyl, and oleyl), disodium edetate dihydrate (EDTA), sodium taurocholate, saponins, cyclodextrins, and cremophor EL), tonicity agents (e.g., dextrose, glycerin, mannitol, potassium chloride, niacinamide, and sodium chloride), gelling agents (e.g., acacia, alginic acid, carmellose sodium (CMC), gelatin, hydroxyethyl cellulose, hydroxypropyl cellulose, magnesium aluminum silicate, methylcellulose, poloxamer 188, poloxamer 407, polyvinyl alcohol, sodium alginate, tragacanth, and xanthan gum), buffering agents (e.g., sodium acetate, ammonium acetate, trisodium citrate, sodium dihydrogen phosphate, boric acid, sodium borate, and trometamol), wetting agents (e.g., benzalkonium chloride, benzethonium chloride, cetylpyridinium chloride, poloxamer 188, poloxamer 407, polyoxyl 40 stearate, polysorbate 20, and polysorbate 40), antioxidants (e.g., ascorbic acid, acetylcysteine, butylated hydroxyanisole (BHA), dibutylhydroxytoluene (BHT), cysteine hydrochloride, dithiothreitol, propyl gallate, sodium metabisulfite, and thiourea), and the like that would be suitable for a specific desired dosage form. Remington's Pharmaceutical Sciences, Edited by Gennaro, Mack Publishing, Easton, Pa., 1995 discloses various carriers used in known technologies for formulation of pharmaceutical compositions and the preparation thereof.

In one embodiment, examples of the utilization method of the invention include, but are not limited to, eye drops. Other examples thereof include dosage modes (administration methods and dosage forms) such as eye ointment, intracameral injection, intravitreal injection, impregnation into a sustained release agent, subconjunctival injection, systemic administration (oral administration, intravenous injection, intraperitoneal administration), and the like.

In another embodiment, EW-7197 or a pharmaceutically acceptable salt thereof, or a solvate thereof can be present in the composition at a concentration of about 0.001% w/v to about 0.5% w/v and preferably about 0.01% w/v to about 0.2% w/v. The effective dose of the medicament of the invention that is effective for treating or preventing a disease state can vary depending on the nature of the specific disease state but can be determined by those skilled in the art with standard clinical technology. Furthermore, an in vitro assay can be used to assist in the identification of the range of optimal dosages as needed. The dosage, although not particularly limited, can be, for example, 0.001, 1, 5, 10, 15, 100, or 1000 mg/kg body weight per dosing or a value within the range of any two of said values. The dosing interval is not particularly limited, but can be, for example, 1 or 2 administrations per 1, 7, 14, 21, or 28 day, or 1 or 2 administrations per day within the range of any two of them. The dosage, number of dosing, dosing interval, dosing period, and dosing method can be appropriately selected depending on the patient's age or body weight, condition, dosing mode, target organ, or the like. For example, the present invention can be used as an eye drop. Further, the medicament of the invention can be injected into the anterior chamber. Further, a therapeutic drug preferably comprises an active ingredient in a therapeutically effective amount, or in an amount effective to exert a desired action. When a therapeutic marker significantly decreases after administration, it can be determined that a therapeutic effect was exerted. An effective dose can be estimated from a dose-response curve obtained from an in vitro or animal model testing system.

Hereinafter, various aspects of the present invention will be described.

In one aspect, the present invention is directed to a method for ameliorating, preventing or treating a disease state mediated by transforming growth factor-β (TFG-β) type I receptor (ALK5) of a subject, wherein the method comprises administering an effective amount of EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof, wherein the EW-7197 (2-fluoro-N-((5-(6-methylpyridin-2-yl)-4-([1,2,4]triazolo[1,5-a]pyridin-6-yl)-1H-imidazol-2-yl)methyl)aniline) is administered in a form of a pharmaceutical composition, which is aqueous solution, and wherein the disease state mediated by ALK5 is selected one from a group of ocular diseases, postsurgical peritoneal adhesions, postsurgical anastomotic strictures, hypertrophic scar, and keloid.

In an embodiment, the composition is for reducing the accumulation of excess extracellular matrix (ECM) in human by inhibiting a TGF-β signaling pathway, for example, inhibiting the phosphorylation of Smad2 or Smad3 by ALK5.

In an embodiment, the ocular disease is selected one from a group of glaucoma, glaucoma filtration surgery bleb failure, intraocular pressure, cataract, posterior capsule opacification (PCO; secondary cataract), corneal haze, wet age-related macular degeneration (AMD), proliferative vitreoretinopathy (PVR), proliferative diabetic retinopathy (PDR), Fuchs' endothelial corneal dystrophy (FECD), and congenital ectopia lentis (CEL).

In an embodiment, EW-7197 or a pharmaceutically acceptable salt thereof, or a solvate thereof is in the composition at a concentration of about 0.001% w/v to about 0.5% w/v.

In an embodiment, EW-7197 or a pharmaceutically acceptable salt thereof, or a solvate thereof is in the composition at a concentration of about 0.01% w/v to about 0.2% w/v.

In an embodiment, said composition further comprises an ophthalmologically acceptable solvent, diluting agent, liquid vehicle, preservative, stabilizer, solubilizer, viscosity enhancer, penetration enhancer, tonicity agent, gelling agent, buffering agent, wetting agent, and/or antioxidant.

In an embodiment, said solubilizer is tyloxapol, polysorbate 80, PEG-40 stearate (MYS-40), PEG-60 hydrogenated castor oil (HCO-60), poloxamer, polyethylene glycol (PEG), PEG/tyloxapol, or PEG/poloxamer.

In an embodiment, the solubilizer is PEG/poloxamer.

In an embodiment, the poloxamer is poloxamer 188 or poloxamer 407.

In an embodiment, the PEG is PEG4000.

In an embodiment, the poloxamer is in the composition at a concentration of about 5% w/v to about 15% w/v based on a total volume of the composition.

In an embodiment, the PEG is in the composition at a concentration of about 20% w/v to about 30% w/v based on a total volume of the composition.

In an embodiment, a weight ratio of the poloxamer and the PEG is 1:2 to 3.

In an embodiment, the composition comprising: EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof at a concentration of about 0.08% w/v to about 0.18% w/v based on a total volume of the composition; the poloxamer at a concentration of about 5% w/v to about 15% w/v based on a total volume of the composition; and the PEG at a concentration of about 20% w/v to about 30% w/v based on a total volume of the composition, wherein a weight ratio of the poloxamer and the PEG is 1:2 to 3.

In an embodiment, composition is an eye drop.

In one aspect, the present invention is directed to a pharmaceutical composition for ameliorating, preventing or treating a disease state mediated by transforming growth factor-β (TFG-β) type I receptor (ALK5) of a subject, wherein the composition is aqueous solution, Wherein the composition comprises EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof at a concentration of about 0.08% w/v to about 0.18% w/v based on a total volume of the composition.

In an embodiment, the composition comprises a poloxamer at a concentration of about 5% w/v to about 15% w/v based on a total volume of the composition; and a PEG at a concentration of about 20% w/v to about 30% w/v based on a total volume of the composition.

Hereinafter, the present invention will be described in more detail with reference to specific examples. However, the following examples are provided only for illustration of the present invention, and should not be construed as limiting the scope of the present invention.

### EXAMPLES

Example 1-8 illustrates formulation development of ophthalmic solution.

### Example 1. Evaluation of Filter Flush Volume

About 0.001% EW-7197 solution was prepared, and the filter flush volume using 2 types of filters was evaluated. About 1 mL each of the filtrate was collected, and the EW-7197 content was measured before and after filtration by HPLC. The recovery rate was calculated from the EW-7197 content measured before filtration. The filters used in the operation are shown in Table 2.

TABLE 2
Filters used for filtration

| | | Pore | Filter | Filtration |
|---|---|---|---|---|
| Filter name | Filter material | size | diameter | area |
| GLchromato-disk | Olefin type | 0.45 μm | 25 mm | 4 cm² |
| 25A | polymer | | | |
| Millipore Sterivex-HV | Polyvinylidene | 0.45 μm | — | 10 cm² |
| 0.45 μm PVDF | Difluoride | | | |
| SVHV010RS | (PVDF) | | | |

The recovery rate of EW-7197 at filtration is shown in Table 3. Generally, the amount of drug absorption decreases when the filtration area becomes smaller. However, the analysis results showed that the amount of EW-7197 absorption of the filter with a larger filtration area (Millipore Sterivex-HV 0.45 μm PVDF SVHV010RS) was lower than that of the filter with a smaller filtration area (GLchromato-disk 25A). The recovery rate of the former filter (Millipore Sterivex-HV 0.45 μm PVDF SVHV010RS) reached an equilibrium after flushing 3 mL of EW-7197 solution. Meanwhile, the recovery rate of the latter filter (GLchromato-disk 25A) reached an equilibrium after flushing 5 mL of EW-7197 solution. This was considered due to the difference in filter material. Both filters can be used by flushing 5 mL or more of EW-7197 solution when the concentration of EW-7197 is not less than 0.00085%.

TABLE 3
EW-7197 Recovery rates at filtration

| Before | | | Recovery |
|---|---|---|---|
| filtration | | Amount of | rate of |
| EW-7197 | | flush solution | EW-7197 |
| conc. (%) | Filter name | (mL) | (%) |
| 0.0008519 | GLchromato-disk 25A | 0 (Before filtration) | 100.0 |
| | Filtration area 4 cm² | 0~1 | 33.5 |
| | | 1~2 | 85.1 |
| | | 2~3 | 96.7 |
| | | 3~4 | 99.3 |
| | | 4~5 | 100.6 |
| | | 5~6 | 100.9 |
| | | 6~7 | 101.4 |
| | | 7~8 | 101.6 |
| | | 8~9 | 101.8 |
| | | 9~10 | 101.9 |
| | Millipore Sterivex filter unit | 0 (Before filtration) | 100.0 |
| | SVHV010RS | 0~1 | 55.1 |
| | Filtration area 10 cm² | 1~2 | 98.1 |
| | | 2~3 | 100.6 |
| | | 3~4 | 101.2 |
| | | 4~5 | 101.4 |
| | | 5~6 | 101.5 |
| | | 6~7 | 101.4 |
| | | 7~8 | 101.7 |
| | | 8~9 | 101.4 |
| | | 9~10 | 101.3 |

### Example 2. Thermal Stability of EW-7197 in Buffer Solutions

The thermal stability of EW-7197 was evaluated in the formulations prepared with various pH values, buffers, and stabilizing agents. The formulations used in the thermal stability test are shown in Table 4. After the preparation of each formulation, the formulation was filtered. The filters used in the operation are shown in Table 2. About 3 mL of the filtrate was filled into a glass ampule, and stored in a thermostatic chamber (25° C.).

TABLE 4
Formulations to test thermastability in buffer solutions

| Ingredients | | | | | | | | | | | | | | |
|---|---|---|---|---|---|---|---|---|---|---|---|---|---|---|
| (g/100 mL)^{*3} | A01 | A02 | A03 | A04 | A05 | A06 | A07 | A13 | A16 | | A19 | | A22 | |
| BW-7197 | 0.001 | 0.001 | 0.001 | 0.001 | 0.001 | 0.001 | 0.001 | 0.001 | 0.001 | | 0.001 | | 0.001 | |
| Sodium | 0.2 | — | — | — | — | — | — | | | | | | | |
| acetate | | | | | | | | | | | | | | |
| hydrate | | | | | | | | | | | | | | |
| Trisodium | — | 0.2 | 0.2 | — | — | — | — | — | — | | — | | — | |
| citrate | | | | | | | | | | | | | | |
| dihydrate | | | | | | | | | | | | | | |
| Sodium | — | — | — | 0.2 | 0.2 | — | — | — | — | | — | | — | |
| dihydrogen | | | | | | | | | | | | | | |
| phosphate | | | | | | | | | | | | | | |
| dihydrate | | | | | | | | | | | | | | |
| Boric acid | — | — | — | — | — | 0.9 | 0.9 | 0.9 | 0.9 | | 1 | | 1 | |
| Sodium | — | — | — | — | — | 0.1 | 0.1 | — | — | | — | | — | |
| borate | | | | | | | | | | | | | | |
| (Bolax) | | | | | | | | | | | | | | |
| Trometamol | — | — | — | — | — | — | — | 0.1 | 0.1 | | — | | — | |
| Disodium | — | — | — | — | — | — | — | — | — | | — | | — | |
| edetate | | | | | | | | | | | | | | |
| dihydrate | | | | | | | | | | | | | | |
| (EDTA) | | | | | | | | | | | | | | |
| Sodium | — | — | — | — | — | — | — | — | — | | — | | — | |
| thiosulfate | | | | | | | | | | | | | | |
| hydrate | | | | | | | | | | | | | | |
| Sodium | q.s. | q.s. | q.s. | q.s. | q.s. | q.s. | q.s. | q.s. | q.s. | | q.s. | | q.s. | |
| hydrate | | | | | | | | | | | | | | |
| Hydrochloric | q.s. | q.s. | q.s. | q.s. | q.s. | q.s. | q.s. | q.s. | q.s. | | q.s. | | q.s. | |
| acid | | | | | | | | | | | | | | |
| pH | 5.5 | 5.5 | 6.5 | 6.5 | 7.5 | 6.5 | 7.5 | 7.5 | 8.0 | | 7.5 | | 8.0 | |
| Ingredient | | | | | | | | | | | | | | |
| (g/100 mL)^{*2} | B01 | B02 | B03 | B04 | B05 | B06 | B07 | C01 | C02 | C03 | C04 | C05 | C06 | C07 |
| BW-7197 | 0.001 | 0.001 | 0.001 | 0.001 | 0.001 | 0.001 | 0.001 | 0.001 | 0.001 | 0.001 | 0.001 | 0.001 | 0.001 | 0.001 |
| Sodium | 0.2 | — | — | — | — | — | — | 0.2 | — | — | — | — | — | — |
| acetate | | | | | | | | | | | | | | |
| hydrate | | | | | | | | | | | | | | |
| Trisodium | — | 0.2 | 0.2 | — | — | — | — | — | 0.2 | 0.2 | — | — | — | — |
| citrate | | | | | | | | | | | | | | |
| dihydrate | | | | | | | | | | | | | | |
| Sodium | — | — | — | 0.2 | 0.2 | — | — | — | — | — | 0.2 | 0.2 | — | — |
| dihydrogen | | | | | | | | | | | | | | |
| phosphate | | | | | | | | | | | | | | |
| dihydrate | | | | | | | | | | | | | | |
| Boric acid | — | — | — | — | — | 0.9 | 0.9 | — | — | — | — | — | 0.9 | 0.9 |
| Sodium | — | — | — | — | — | 0.1 | 0.1 | — | — | — | — | — | 0.1 | 0.1 |
| borate | | | | | | | | | | | | | | |
| (Bolax) | | | | | | | | | | | | | | |
| Trometamol | — | — | — | — | — | — | — | — | — | — | — | — | — | — |
| Disodium | 0.01 | 0.01 | 0.01 | 0.01 | 0.01 | 0.01 | 0.01 | — | — | — | — | — | — | — |
| edetate | | | | | | | | | | | | | | |
| dihydrate | | | | | | | | | | | | | | |
| (EDTA) | | | | | | | | | | | | | | |
| Sodium | — | — | — | — | — | — | — | 0.01 | 0.01 | 0.01 | 0.01 | 0.01 | 0.01 | 0.01 |
| thiosulfate | | | | | | | | | | | | | | |
| hydrate | | | | | | | | | | | | | | |
| Sodium | q.s | q.s | q.s | q.s | q.s | q.s | q.s | q.s | q.s | q.s | q.s | q.s | q.s | q.s |
| hydrate | | | | | | | | | | | | | | |
| Hydrochloric | q.s | q.s | q.s | q.s | q.s | q.s | q.s | q.s | q.s | q.s | q.s | q.s | q.s | q.s |
| acid | | | | | | | | | | | | | | |
| pH | 5.5 | 5.5 | 6.5 | 6.5 | 7.5 | 6.5 | 7.5 | 5.5 | 5.5 | 6.5 | 6.5 | 7.5 | 6.5 | 7.5 |

^{*2}Each formulation was prepared at the density of 1 g/mL.

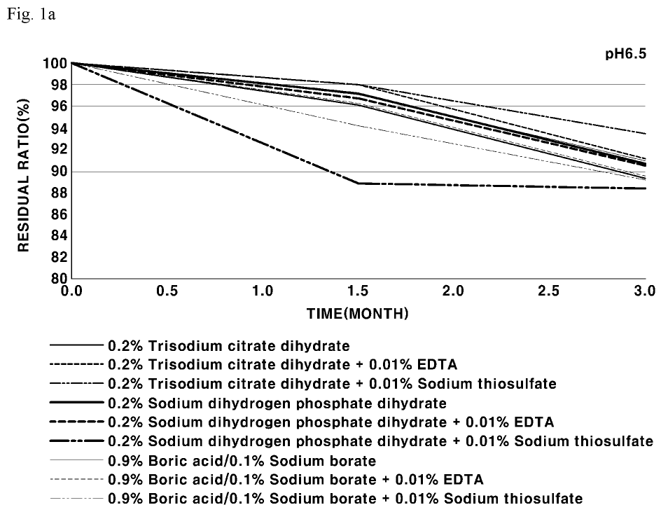

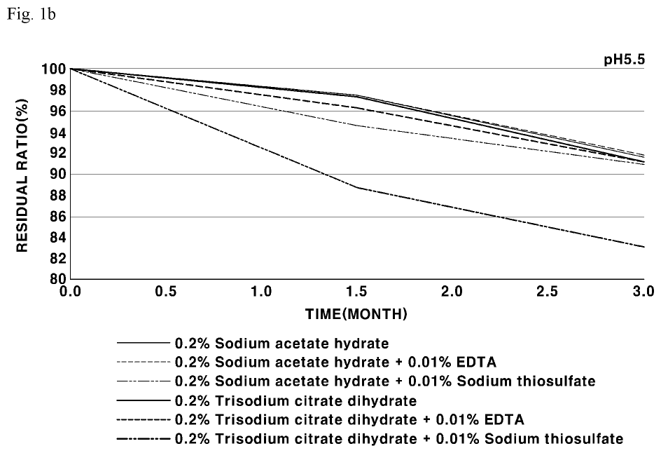

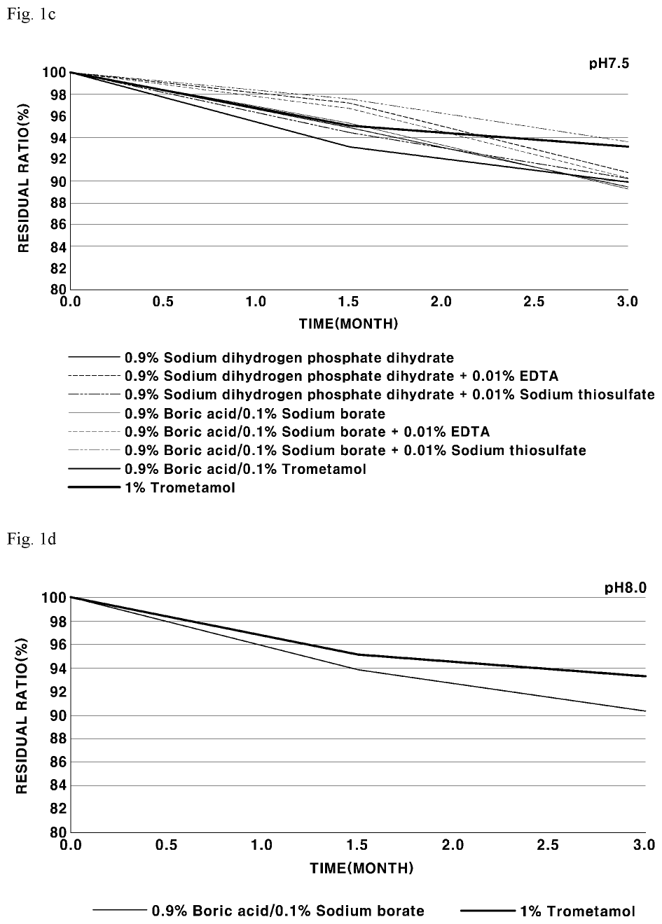

The results are shown in Table 5. The results of the thermal stability test at various pH values are shown in FIG. 1. When the stability was measured with respective buffers, the results were different depending on pH values and the presence of stabilizing agent. Regarding the formulations with no stability agents (Series A), the formulations at low pH values had a slightly higher stability when using the same buffer. However, regarding the boric acid/trometamol formulations (A13 and A16) and the trometamol formulations (A19 and A22), the thermal stability of EW-7197 did not differ by pH values. Among the evaluated formulations, the trometamol formulations (A19 and A22) had the highest thermal stability of EW-7197. Regarding the formulations that contain disodium edetate dihydrate (EDTA) (Series B), the thermal stability of EW-7197 did not improve by the addition of EDTA in most of the formulations. A trend of slight improvement of the thermal stability of EW-7197 was shown in the formulations at pH7.5 (B05 and B07), when compared to the formulations that do not contain EDTA. Regarding the formulations that contains sodium thiosulfate hydrate (sodium thiosulfate) (Series C), the thermal stability of EW-7197 decreased or did not change by the addition of sodium thiosulfate for most of the formulations. Especially, the formulations at low pH values were unstable when using the same buffer. While the stability decreased in many formulations, the thermal stability of EW-7197 improved by the addition of sodium thiosulfate in the formulation that contains sodium citrate at pH6.5 (C03) and the formulation that contain boric acid and sodium borate at pH7.5 (C07). Since sodium thiosulfate is decomposed in acidic solution, it is desirable to use the boric acid and sodium borate buffer at not less than pH7.5.

TABLE 5
Thermal stability of EW-7197 in buffers (25° C.)

| | | | Sodium | | | EW-7197 | Residual ratio (%) | | |
|---|---|---|---|---|---|---|---|---|---|
| Formulation | | EDTA | thiosulfate | Storage | | conc. (%) | | 25° C. | 25° C. |
| number | Buffer solutions | conc. (%) | conc. (%) | container | pH | of Initial | Initial | 1.5 months | 3 months |
| A01 | 0.2% Sodium acetate | — | — | GA | 5.5 | 0.0008638 | 100.0 | 97.6 | 91.7 |
| | hydrate | | | | | | | | |
| A02 | 0.2% Trisodium | — | — | GA | | 0.0008680 | 100.0 | 97.4 | 91.3 |
| A03 | citrate dihydrate | — | — | GA | 6.5 | 0.0008750 | 100.0 | 96.1 | 89.4 |
| A04 | 0.2% Sodium | — | — | GA | | 0.0008517 | 100.0 | 97.2 | 90.7 |
| A05 | dihydrogen | — | — | GA | 7.5 | 0.0008594 | 100.0 | 94.9 | 89.5 |
| | phosphate-dihydrate | | | | | | | | |
| A06 | 0.9% Boric acid | — | — | GA | 6.5 | 0.0008537 | 100.0 | 97.1 | 91.0 |
| A07 | 0.1% Sodium borate | — | — | GA | 7.5 | 0.0008724 | 100.0 | 95.4 | 89.3 |
| A13 | 0.9% Boric acid | — | — | GA | 7.5 | 0.0008304 | 100.0 | 93.2 | 90.0 |
| A16 | 0.1% Trometamol | — | — | GA | 8.0 | 0.0008643 | 100.0 | 93.9 | 90.4 |
| A19 | 0.1% Trometamol | — | — | GA | 7.5 | 0.0008730 | 100.0 | 95.2 | 93.2 |
| A22 | | — | — | GA | 8.0 | 0.0008729 | 100.0 | 95.2 | 93.4 |
| B01 | 0.2% Sodium acetate | 0.01 | — | GA | 5.5 | 0.0008700 | 100.0 | 97.5 | 91.9 |
| | hydrate | | | | | | | | |
| B02 | 0.2% Trisodium | 0.01 | — | GA | | 0.0008672 | 100.0 | 96.4 | 91.3 |
| B03 | citrate dihydrate | 0.01 | — | GA | 6.5 | 0.0008569 | 100.0 | 98.0 | 91.2 |
| B04 | 0.2% Sodium | 0.01 | — | GA | | 0.0008638 | 100.0 | 96.8 | 90.6 |
| B05 | dihydrogen | 0.01 | — | GA | 7.5 | 0.0008521 | 100.0 | 97.2 | 90.8 |
| | phosphate-dihydrate | | | | | | | | |
| B06 | 0.9% Boric acid | 0.01 | — | GA | 6.5 | 0.0008749 | 100.0 | 96.3 | 89.6 |
| B07 | 0.1% Sodium borate | 0.01 | — | GA | 7.5 | 0.0008720 | 100.0 | 96.7 | 90.3 |
| C01 | 0.2% Sodium acetate | — | 0.01 | GA | 5.5 | 0.0008612 | 100.0 | 94.7 | 91.0 |
| | hydrate | | | | | | | | |
| C02 | 0.2% Trisodium | — | 0.01 | GA | | 0.0008732 | 100.0 | 88.8 | 83.2 |
| C03 | citrate dihydrate | — | 0.01 | GA | 6.5 | 0.0008755 | 100.0 | 98.0 | 93.5 |
| C04 | 0.2% Sodium | — | 0.01 | GA | | 0.0008628 | 100.0 | 88.9 | 88.4 |
| C05 | dihydrogen | — | 0.01 | GA | 7.5 | 0.0008610 | 100.0 | 94.5 | 90.3 |
| | phosphate-dihydrate | | | | | | | | |
| C06 | 0.9% Boric acid | — | 0.01 | GA | 6.5 | 0.0008689 | 100.0 | 94.2 | 89.2 |
| C07 | 0.1% Sodium borate | — | 0.01 | GA | 7.5 | 0.0008670 | 100.0 | 97.6 | 93.7 |

### Example 3. Photostability of EW-7197 in Buffer Solutions

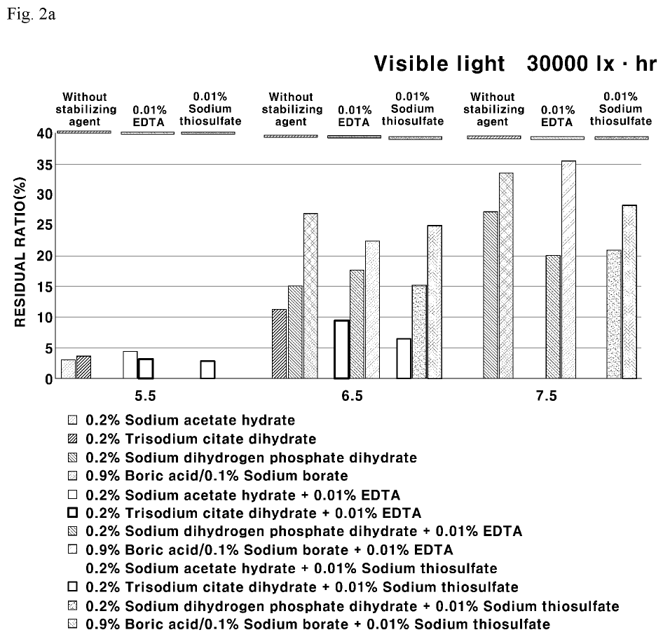

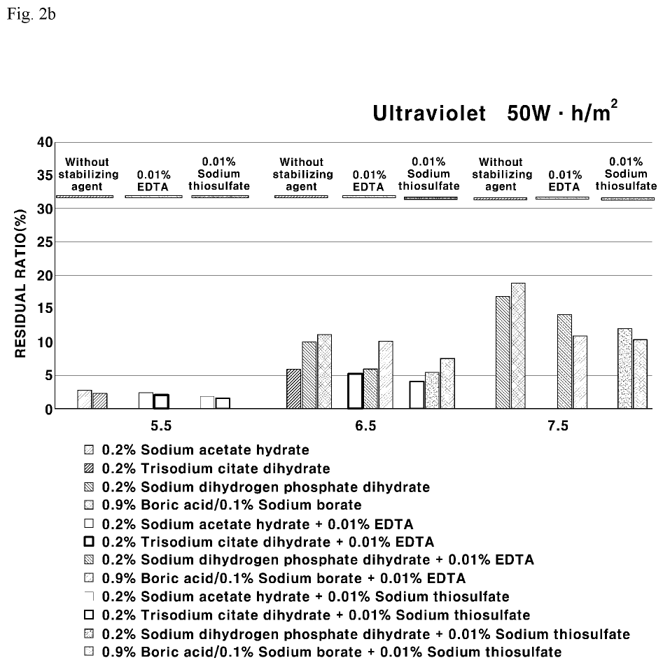

The photostability of EW-7197 was evaluated in the formulations prepared with various pH values, buffers, and stabilizing agent. The formulations used in the photostability test are the same formulations (excluding A13, A16, A19, and A22) used for evaluation of thermal stability shown in Table 4. After preparation, each formulation was filtered. The filters used in the operation were shown in Table 2. About 3 mL of the filtrate was filled in a glass ampule, and illuminated with ultraviolet light of 50 W·h/m² and visible light of 300,000 lx·hr by a photostability test chamber. The results are shown in Table 6 and FIG. 2. In the results of the respective buffers, the photostability of EW-7197 increased when the pH value increased. Meanwhile, EDTA and sodium thiosulfate did not have the photostabilizing effect on EW-7197 against the visible and ultraviolet light. Generally, illumination under white fluorescent lighting is about 1000 lx per hour. Based on the above results, it is considered that 1-5% of EW-7197 is decomposed by light when EW-7197 solution is left in a room for a whole day under fluorescent lighting. Therefore, EW-7197 needs to be stored in light shielded conditions.

TABLE 6
Photostability of EW-7197 in buffers (ultraviolet and visible light)

| | | | Sodium | | | BW-7197 | Residual ratio (%) | | |
|---|---|---|---|---|---|---|---|---|---|
| Formulation | | EDTA | thiosulfate | Storage | | conc. (%) | | Ultraviolet | Visible light |
| number | Buffer solutions | conc. (%) | conc. (%) | container | pH | of Initial | Inital | 50 W · h/m² | 30000 1× · hr |
| A01 | 0.2% Sodium acetate | — | — | GA | 5.5 | 0.0008638 | 100.0 | 2.8 | 3.0 |
| | hydrate | | | | | | | | |
| A02 | 0.2% Trisodium | — | — | GA | | 0.0008680 | 100.0 | 2.3 | 3.5 |
| A03 | citrate dihydrate | — | — | GA | 6.5 | 0.0008750 | 100.0 | 6.0 | 11.2 |
| A04 | 0.2% Sodium | — | — | GA | | 0.0008517 | 100.0 | 10.1 | 15.1 |
| A05 | dihydrogen | — | — | GA | 7.5 | 0.0008594 | 100.0 | 16.8 | 27.2 |
| | phosphate-dihydrate | | | | | | | | |
| A06 | 0.9% Boric acid | — | — | GA | 6.5 | 0.0008537 | 100.0 | 11.1 | 27.0 |
| A07 | 0.1% Sodium borate | — | — | GA | 7.5 | 0.0008724 | 100.0 | 18.8 | 33.6 |
| B01 | 0.2% Sodium acetate | 0.01 | — | GA | 5.5 | 0.0008700 | 100.0 | 2.4 | 4.4 |
| | hydrate | | | | | | | | |
| B02 | 0.2% Trisodium | 0.01 | — | GA | | 0.0008672 | 100.0 | 2.2 | 3.1 |
| B03 | citrate dihydrate | 0.01 | — | GA | 6.5 | 0.0008569 | 100.0 | 5.3 | 9.4 |
| B04 | 0.2% Sodium | 0.01 | — | GA | | 0.0008638 | 100.0 | 6.0 | 17.6 |
| B05 | dihydrogen | 0.01 | — | GA | 7.5 | 0.0008521 | 100.0 | 14.1 | 20.2 |
| | phosphate-dihydrate | | | | | | | | |
| B06 | 0.9% Boric acid | 0.01 | — | GA | 6.5 | 0.0008749 | 100.0 | 10.2 | 22.4 |
| B07 | 0.1% Sodium borate | 0.01 | — | GA | 7.5 | 0.0008720 | 100.0 | 11.0 | 35.5 |
| C01 | 0.2% Sodium acetate | — | 0.01 | GA | 5.5 | 0.0008612 | 100.0 | 1.9 | 3.3 |
| | hydrate | | | | | | | | |
| C02 | 0.2% Trisodium | — | 0.01 | GA | | 0.0008732 | 100.0 | 1.7 | 2.9 |
| C03 | citrate dihydrate | — | 0.01 | GA | 6.5 | 0.0008755 | 100.0 | 4.1 | 6.4 |
| C04 | 0.2% Sodium | — | 0.01 | GA | | 0.0008628 | 100.0 | 5.5 | 15.3 |
| C05 | dihydrogen | — | 0.01 | GA | 7.5 | 0.0008610 | 100.0 | 12.0 | 20.9 |
| | phosphate-dihydrate | | | | | | | | |
| C06 | 0.9% Boric acid | — | 0.01 | GA | 6.5 | 0.0008689 | 100.0 | 7.6 | 24.9 |
| C07 | 0.1% Sodium borate | — | 0.01 | GA | 7.5 | 0.0008670 | 100.0 | 10.4 | 28.3 |

### Example 4. Exploring for Solubilizing Agents

As shown in Table 1, the solubility of EW-7197 is significantly low within an acceptable pH range for ophthalmic solution development, various solubilizing agents were evaluated. The formulations used in the evaluation of the solubilization study are shown in Table 7. The buffer solution used in this study was 0.9% boric acid/0.1% sodium borate (pH7.5). Each formulation was prepared at 30 g scale, and after adding EW-7197, the formulations were suspended at 4° C. and 25° C. After confirming that the formulations were suspended at each temperature, each formulation was filtered. The EW-7197 content of the filtrate was measured by HPLC. The filters used in the operation are shown in Table 2.

TABLE 7
Formulations used in exploring of solubilizing agents

| Ingredients (g/100 mL)^{*3} | D00 | D01 | D02 | D03 | D04 | D05 | D06 | D07 | D08 | D09 | D10 | D11 | D12 | D16 |
|---|---|---|---|---|---|---|---|---|---|---|---|---|---|---|
| Boric acid | 0.9 | 0.9 | 0.9 | 0.9 | 0.9 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 | 0.1 | 0.1 |
| Polysorbate 80 | — | 0.2 | 0.5 | 1 | — | — | — | — | — | — | — | — | — | — |
| Tyloxapol | — | — | — | — | 0.2 | 0.5 | 1 | — | — | — | — | — | — | — |
| PEG-40 Stearate (MYS-40) | — | — | — | — | — | — | — | 0.2 | 0.5 | 1 | — | — | — | — |
| PEG-60 hydrogenated Castor | — | — | — | — | — | — | — | — | — | — | 0.2 | 0.5 | 1 | — |
| oil (HCO-60) | | | | | | | | | | | | | | |
| Benzalkonium chloride (BAK) | — | — | — | — | — | — | — | — | — | — | — | — | — | 0.01 |
| Sodium hydrate | q.s. | q.s. | q.s. | q.s. | q.s. | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 | 7.5 | 7.5 | 7.5 | 7.5 | 7.5 |
| Ingredients (g/100 mL)^{*2} | D17 | D18 | D19 | D20 | D21 | D22 | D23 | D24 | D25 | D26 | D27 | D28 | D29 | D30 |
| Boric acid | 0.9 | 0.9 | 0.9 | 0.9 | 0.9 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 | 0.1 | 0.1 |
| Metolose SM-25 (MC SM-25) | 0.5 | — | — | — | — | — | — | — | — | — | — | — | — | — |
| Hypromellose 60SH-50 | — | 0.5 | — | — | — | — | — | — | — | — | — | — | — | — |
| (HPMC 60SH-50) | | | | | | | | | | | | | | |
| Hypromellose 60SH-4000 | — | — | 0.5 | — | — | — | — | — | — | — | — | — | — | — |
| (HPMC 60SH-4000) | | | | | | | | | | | | | | |
| Carmellose sodium (CMC) | — | — | — | 0.5 | — | — | — | — | — | — | — | — | — | — |
| Povidone K-30 (PVP) | — | — | — | — | 0.5 | — | — | — | — | — | — | — | — | — |
| Lipidure-PMB (Lipidure) | — | — | — | — | — | 0.5 | — | — | — | — | — | — | — | — |
| Polyethylene glycol 400 | — | — | — | — | — | — | 1 | — | — | — | — | — | — | — |
| (PEG400) | | | | | | | | | | | | | | |
| Polyethylene glycol 4000 | — | — | — | — | — | — | — | 1 | 4 | 10 | — | — | — | — |
| (PEG4000) | | | | | | | | | | | | | | |
| Propylene glycol (PG) | — | — | — | — | — | — | — | — | — | — | 1 | — | — | — |
| Glycerin | — | — | — | — | — | — | — | — | — | — | — | 1 | — | — |
| Niacinamide | — | — | — | — | — | — | — | — | — | — | — | — | 1 | 2 |
| Sodium hydrate | q.s | q.s | q.s | q.s | q.s | q.s | q.s | q.s | q.s | q.s | q.s | q.s | q.s | q.s |
| Hydrochloric acid | q.s | q.s | q.s | q.s | q.s | q.s | q.s | q.s | q.s | q.s | q.s | q.s | q.s | q.s |
| pH | 7.5 | 7.5 | 7.5 | 7.5 | 7.5 | 7.5 | 7.5 | 7.5 | 7.5 | 7.5 | 7.5 | 7.5 | 7.5 | 7.5 |

| Ingredients (g/100 mL)^{*2} | E00 | E08 | E09 | E10 | E11 | E12 | E13 | E14 |
|---|---|---|---|---|---|---|---|---|
| Boric acid | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 |
| Tyloxapol | — | 0.3 | 0.5 | 0.5 | 0.5 | 1 | 1 | 1 |
| Polyethylene glycol 4000 | 4 | 4 | 4 | — | 4 | 4 | — | 4 |
| (PEG4000) | | | | | | | | |
| Niacinamide | 2 | 2 | — | 2 | 2 | — | 2 | 2 |
| Sodium hydrate | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 |

^{*3}Each formulation was prepared at the density of 1 g/mL.

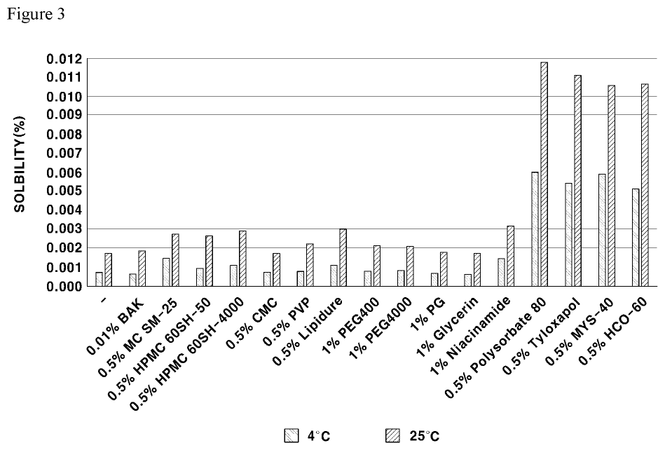

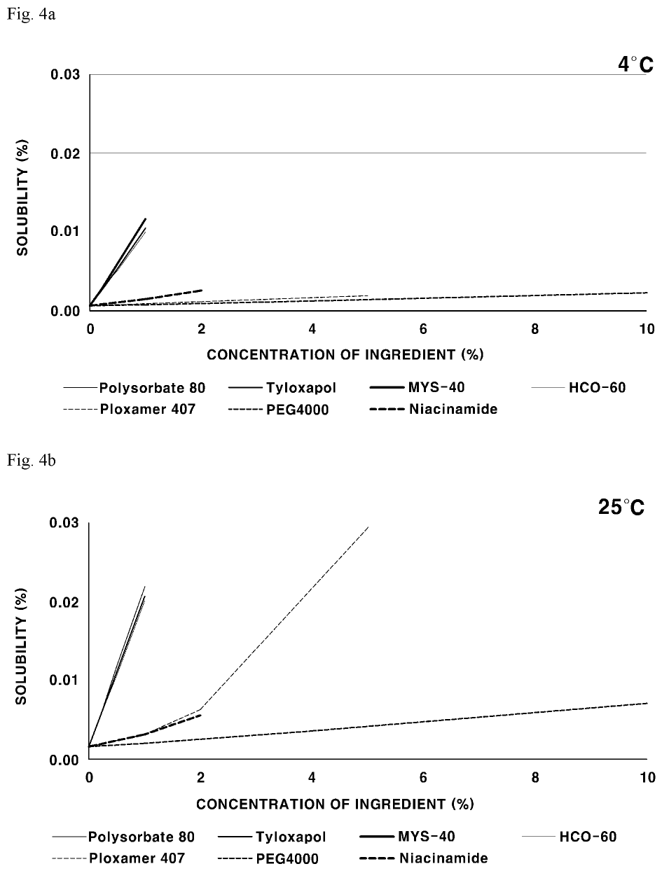

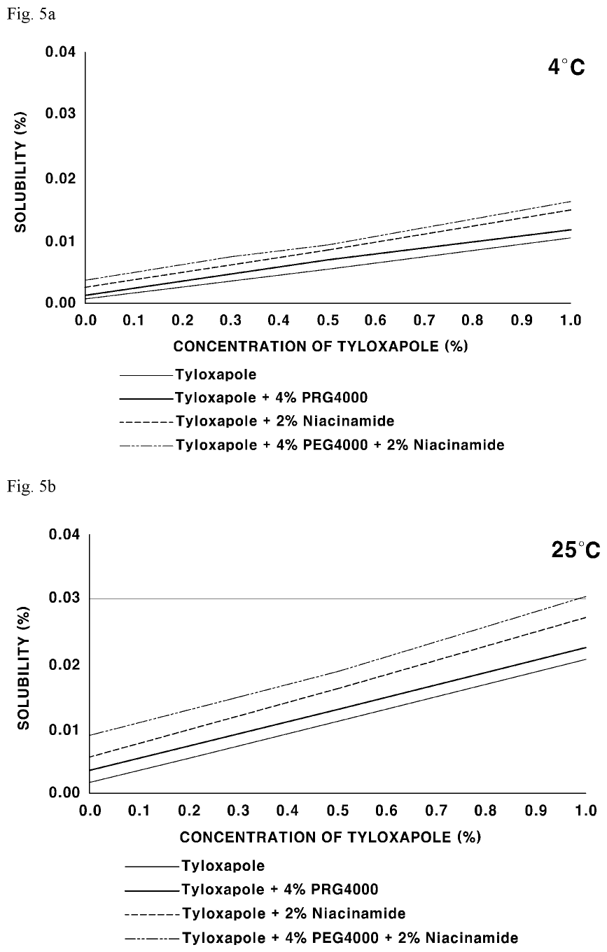

The evaluation results of the solubility of EW-7197 when solubilizing agents were added are shown in Table 8. The solubility of EW-7197 with respective solubilizing agents is shown in FIG. 3, the solubility of EW-7197 with respective concentrations of surfactants, PEG4000, and niacinamide is shown in FIG. 4, and the solubility of EW-7197 when all of tyloxapol, PEG4000, and niacinamide were used is shown in FIG. 5. Water-soluble polymers did not have solubilizing effect on EW-7197. Among various tonicity agents, only niacinamide has a slight solubilizing effect on EW-7197. As shown in FIG. 4, the evaluation results on various surfactants show that each surfactant has a certain level of solubilizing effect, and the solubility of EW-7197 with respective surfactants was at the same level. When adding 1% of each surfactant, about 0.01% of EW-7197 was dissolved at 4° C. and about 0.02% was dissolved at 25° C. As shown in FIG. 5, when tyloxapol was used with PEG4000 and niacinamide, the solubility of EW-7197 at 4° C. and 25° C. was about 1.5 times higher than the solubility when tyloxapol was used solely. Therefore, when developing a formulation with 1% tyloxapol, 4% PEG4000, and 2% niacinamide, the upper limit of concentration of EW-7197 is considered to be about 0.015%.

TABLE 8
Solubility with various solubilizing agents (4° C. and 25° C.)

| | | | | 4° C. | 25° C. |
|---|---|---|---|---|---|
| | Solubilizing | | | EW-7197 | EW-7197 |
| Formulation | agent | Solubilizing | | solubility | solubility |
| number | conc. (%) | agents | pH | (%) | (%) |
| D00 | — | | 7.5 | 0.0007277 | 0.0017161 |
| D16 | 0.01 | BAK | 7.5 | 0.0006589 | 0.0018652 |
| D17 | 0.5 | MC SM-25 | 7.5 | 0.0014722 | 0.0027500 |
| D18 | 0.5 | HPMC | 7.5 | 0.0009430 | 0.0026433 |
| | | 60SH-50 | | | |
| D19 | 0.5 | HPMC | 7.5 | 0.0010892 | 0.0029175 |
| | | 60SH-4000 | | | |
| D20 | 0.5 | CMC | 7.5 | 0.0007288 | 0.0017653 |
| D21 | 0.5 | PVP | 7.5 | 0.0007750 | 0.0022091 |
| D22 | 0.5 | Lipidure | 7.5 | 0.0011309 | 0.0030467 |
| D23 | 1 | PEG400 | 7.5 | 0.0007844 | 0.0021288 |
| D24 | 1 | PEG4000 | 7.5 | 0.0008102 | 0.0020978 |
| D25 | 4 | PEG4000 | 7.5 | 0.0012900 | 0.0035989 |
| D26 | 10 | PEG4000 | 7.5 | 0.0023204 | 0.0071584 |
| D27 | 1 | PG | 7.5 | 0.0006895 | 0.0017944 |
| D28 | 1 | Glycerin | 7.5 | 0.0006457 | 0.0017234 |
| D29 | 1 | Niacinamide | 7.5 | 0.0014587 | 0.0032068 |
| D30 | 2 | Niacinamide | 7.5 | 0.0025646 | 0.0056196 |
| E00 | 4 | PEG4000 | 7.5 | 0.0037331 | 0.0089662 |
| | 2 | Niacinamide | 7.5 | | |
| D01 | 0.2 | Polysorbate | 7.5 | 0.0027827 | 0.0055695 |
| | | 80 | | | |
| D02 | 0.5 | Polysorbate | 7.5 | 0.0059988 | 0.0117987 |
| | | 80 | | | |
| D03 | 1 | Polysorbate | 7.5 | 0.0114852 | 0.0218575 |
| | | 80 | | | |
| D04 | 0.2 | Tyloxapol | 7.5 | 0.0025618 | 0.0054409 |
| D05 | 0.5 | Tyloxapol | 7.5 | 0.0054477 | 0.0111277 |
| D06 | 1 | Tyloxapol | 7.5 | 0.0103369 | 0.0206627 |
| E01 | 0.3 | Tyloxapol | 7.5 | 0.0073965 | 0.0148636 |
| | 4 | PEG4000 | 7.5 | | |
| | 2 | Niacinamide | 7.5 | | |
| E02 | 0.5 | Tyloxapol | 7.5 | 0.0068873 | 0.0130107 |
| | 4 | PEG4000 | 7.5 | | |
| E03 | 0.5 | Tyloxapol | 7.5 | 0.0084560 | 0.0161941 |
| | 2 | Niacinamide | 7.5 | | |
| E04 | 0.5 | Tyloxapol | 7.5 | 0.0092463 | 0.0188141 |
| | 4 | PEG4000 | 7.5 | | |
| | 2 | Niacinamide | 7.5 | | |
| E05 | 1 | Tyloxapol | 7.5 | 0.0116266 | 0.0225032 |
| | 4 | PEG4000 | 7.5 | | |
| E06 | 1% | Tyloxapol | 7.5 | 0.0148310 | 0.0271268 |
| | 2% | Niacinamide | 7.5 | | |
| E07 | 1% | Tyloxapol | 7.5 | 0.0161264 | 0.0303901 |
| | 4% | PEG4000 | 7.5 | | |
| | 2% | Niacinamide | 7.5 | | |
| D07 | 0.2 | MYS-40 | 7.5 | 0.0027652 | 0.0052257 |
| D08 | 0.5 | MYS-40 | 7.5 | 0.0059463 | 0.0106046 |
| D09 | 1 | MYS-40 | 7.5 | 0.0115629 | 0.0200239 |
| D10 | 0.2 | HCO-60 | 7.5 | 0.0028038 | 0.0055497 |
| D11 | 0.5 | HCO-60 | 7.5 | 0.0051621 | 0.0106829 |
| D12 | 1 | HCO-60 | 7.5 | 0.0099208 | 0.0201615 |

### Example 5. Thermal Stability of EW-7197 in Formulations Containing Various Surfactants

The thermal stability of EW-7197 in the formulations containing various surfactants (tyloxapol, polysorbate 80, PEG-40 stearate (MYS-40), PEG-60 hydrogenated castor oil (HCO-60)) was evaluated as shown in Table 9. In addition, dibutylhydroxytoluene (BHT) was added to the formulations each to evaluate the effect of BHT on the thermal stability of EW-7197. After the preparation of each formulation, the formulation was filtered. The filters used in the operation are shown in Table 2. The filtrate was filled into a glass ampule, and stored in a thermostat bath (40° C.).

TABLE 9
Formulations with various surfactants for thermal stability test

| Ingredient (g/100 mL)^{*4} | F01 | F02 | F12 | F22 | F23 | F24 | F25 | F26 | F27 | F28 | F29 | F30 |
|---|---|---|---|---|---|---|---|---|---|---|---|---|
| EW-7197 | 0.01 | 0.01 | 0.01 | 0.01 | 0.01 | 0.01 | 0.01 | 0.01 | 0.01 | 0.01 | 0.01 | 0.01 |
| Boric acid | 0.9 | 0.9 | 0.9 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 |
| Polysorbate 80 | — | — | — | 0.5 | 1 | 1 | — | — | — | — | — | — |
| Tyloxapol | 0.5 | 1 | 1 | — | — | — | — | — | — | — | — | — |
| PEG-40 Stearate (MYS-40) | — | — | — | — | — | — | 0.5 | 1 | 1 | — | — | — |
| PEG-60 hydrogenated Castor | — | — | — | — | — | — | — | — | — | 0.5 | 1 | 1 |
| oil (HCO-60) | | | | | | | | | | | | |
| Dibutyl hydroxyl toluene | — | — | 0.005 | — | — | 0.005 | — | — | 0.005 | — | — | 0.005 |
| (BHT) | | | | | | | | | | | | |
| Sodium hydrate | q.s | q.s. | q.s. | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s | q.s. | q.s. | q.s | q.s. |
| pH | 7.5 | 7.5 | 7.5 | 7.5 | 7.5 | 7.5 | 7.5 | 7.5 | 7.5 | 7.5 | 7.5 | 7.5 |

^{*4}Each formulation was prepared at the density of 1 g/mL.

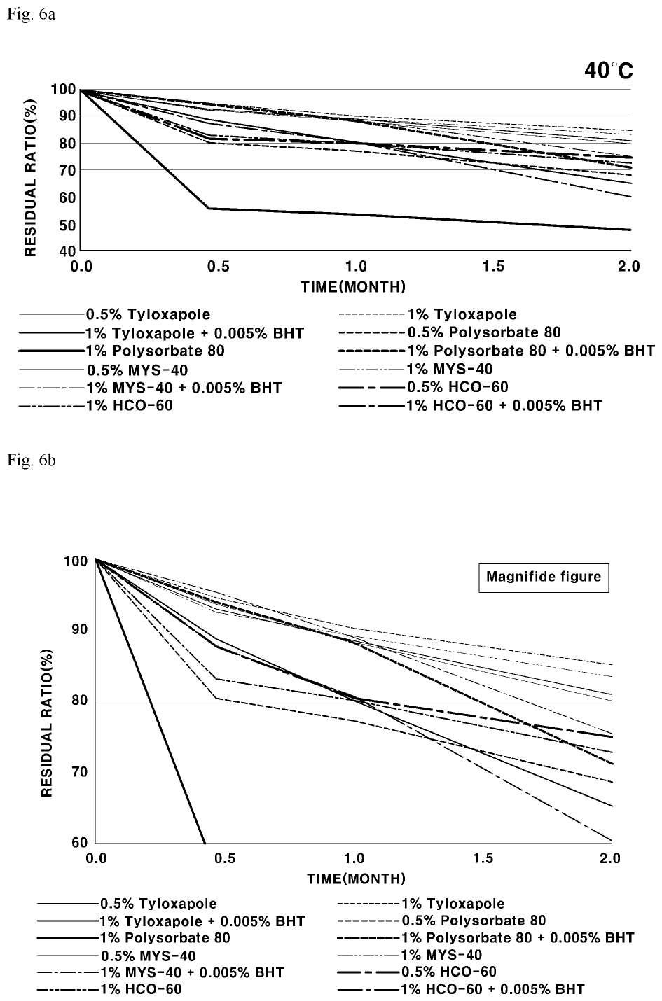

The results are shown in Table 10 and FIG. 6. Among 4 types of surfactants, tyloxapol and MYS-40 showed the equivalent results with the highest thermal stability. HCO-60 and polysorbate 80 were low in thermal stability, especially low for polysorbate 80. The thermal stability of tyloxapol and MYS-40 increased as their concentrations increased. Regarding the 3 types of surfactants, except for polysorbate 80, the addition of BHT caused a decrease of the thermal stability of EW-7197. Meanwhile, the addition of BHT significantly improved the thermal stability of EW-7197 in polysorbate 80, but the stability of EW-7197 was still lower than the formulations of tyloxapol and MYS-40 that did not contain BHT. Based on the above, among the 4 types of surfactants, either tyloxapol or MYS-40 was considered the best to be used as a solubilizing agent.

TABLE 10
Thermal stability with various surfactants (40° C.)

| | | | | | | Residual ratio (%) | | | |
|---|---|---|---|---|---|---|---|---|---|
| Formulation | Surfactant | | BHT | | EW-7197 | | 40° C. | 40° C. | 40° C. |
| number | Conc. (%) | Name | Conc. (%) | pH | Conc. (%) | Initial | 1 weeks | 1 month | 2 months |
| F01 | 0.5 | Tyloxapol | — | 7.5 | 0.01 | 100.0 | 92.9 | 88.5 | 81.1 |
| F02 | 1 | | — | 7.5 | 0.01 | 100.0 | 94.5 | 90.3 | 85.1 |
| F12 | 1 | | 0.005 | 7.5 | 0.01 | 100.0 | 88.8 | 80.1 | 65.4 |
| F22 | 0.5 | Polysorbate80 | — | 7.5 | 0.01 | 100.0 | 80.5 | 77.3 | 68.7 |
| F23 | 1 | | — | 7.5 | 0.01 | 100.0 | 55.9 | 53.7 | 48.3 |
| F24 | 1 | | 0.005 | 7.5 | 0.01 | 100.0 | 94.0 | 88.3 | 71.3 |
| F25 | 0.5 | MYS-40 | — | 7.5 | 0.01 | 100.0 | 93.7 | 88.3 | 80.0 |
| F26 | 1 | | — | 7.5 | 0.01 | 100.0 | 92.5 | 89.3 | 83.5 |
| F27 | 1 | | 0.005 | 7.5 | 0.01 | 100.0 | 95.3 | 89.0 | 75.5 |
| F28 | 0.5 | HCO-60 | — | 7.5 | 0.01 | 100.0 | 87.7 | 80.4 | 75.1 |
| F29 | 1 | | — | 7.5 | 0.01 | 100.0 | 83.2 | 80.2 | 72.9 |
| F30 | 1 | | 0.005 | 7.5 | 0.01 | 100.0 | 87.7 | 80.7 | 60.5 |

### Example 6. Thermal Stability of EW-7197 in Formulations Containing Various Stabilizing Agents

The thermal stability of EW-7197 in the formulations solubilized with tyloxapol (hereinafter, referred to as the tyloxapol formulations) containing various stabilizing agents was evaluated as shown in Table 11. After the preparation of each formulation, the formulation was filtered. The filters used in the operation are shown in Table 2. For F02, F03, F12, F05, F09, F10 and F11, each filtrate was filled into a glass ampule, and stored in a thermostat bath (40° C.). For F02 and F03, each filtrate was filled into glass ampules and polyethylene ophthalmic bottles (PE bottles), and stored in a thermostatic chamber (40° C.). For F120 and F125, each filtrate was filled into a PE bottle only, and stored in thermostatic chambers (25° C. and 40° C.).

TABLE 11
Thermal stability test formulations with various stabilizing agents

| Ingredient (g/100 mL)^{*5} | F02 | F03 | F12 | F05 | F09 | F10 | F11 | F120 | F128 |
|---|---|---|---|---|---|---|---|---|---|
| EW-7197 | 0.01 | 0.01 | 0.01 | 0.01 | 0.01 | 0.01 | 0.01 | 0.015 | 0.015 |
| Boric acid | 0.9 | 0.9 | 0.9 | 0.9 | 0.9 | 0.9 | 0.9 | 1.6 | 1.6 |
| Sodium borate (Bolax) | 0.1 | 0.1 | 0.1 | 0.1 | 0.1 | 0.1 | 0.1 | 0.3 | 0.3 |
| Tyloxapol | 1 | 1 | 1 | 1 | 1 | 1 | 1 | 1 | 1 |
| Disodium edetate | — | 0.01 | — | — | — | — | — | 0.01 | 0.01 |
| dihydrate (EDTA) | | | | | | | | | |
| Dibutyl hydroxyl toluene | — | — | 0.005 | — | — | — | — | — | — |
| (BHT) | | | | | | | | | |
| Mannitol | — | — | — | 0.1 | — | — | — | — | — |
| Polyethylene glycol 4000 | — | — | — | — | 4 | — | — | — | — |
| (PEG400) | | | | | | | | | |
| Niacinamide | — | — | — | — | — | 2 | — | — | — |
| Hypromellose 60SH-4000 | — | — | — | — | — | — | 0.02 | — | — |
| (HPMC 60SH-4000) | | | | | | | | | |
| Sodium thiosulfate hydrate | — | — | — | — | — | — | — | — | 0.1 |
| Sodium hydrate | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 |

^{*5}Each formulation was prepared at the density of 1 g/mL.

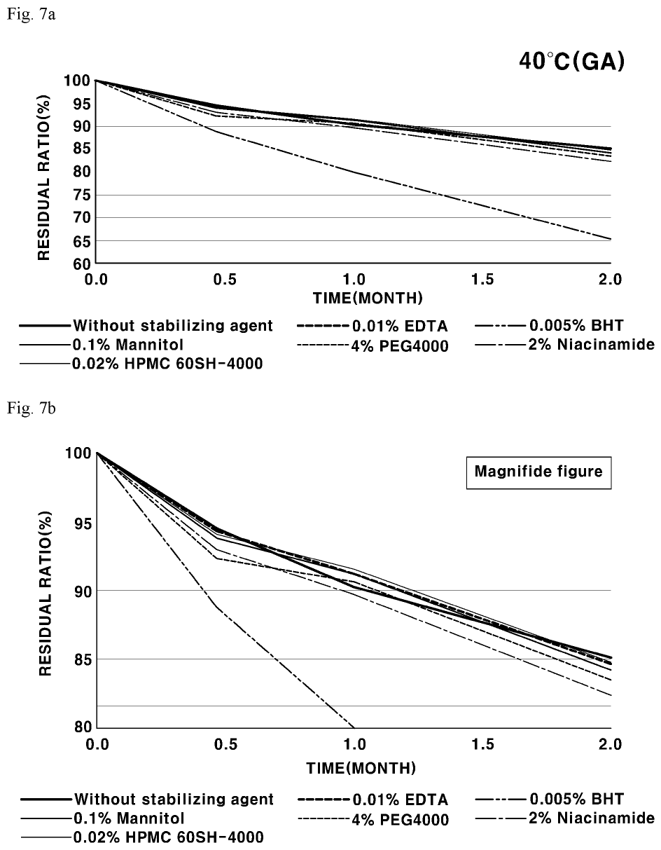

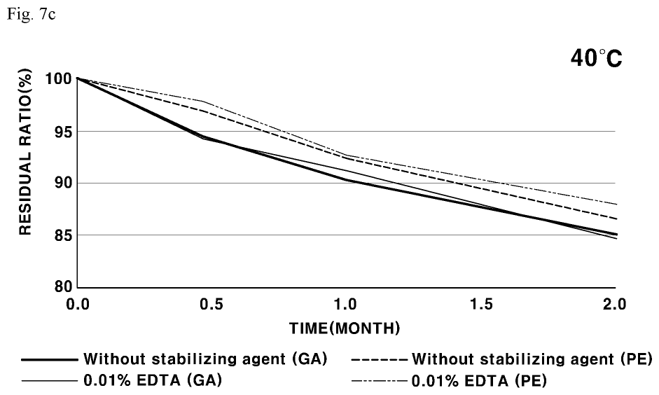

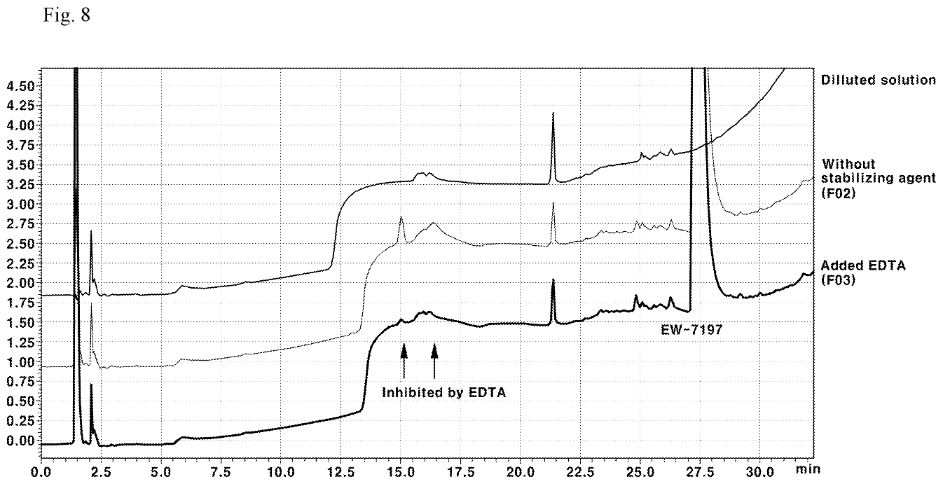

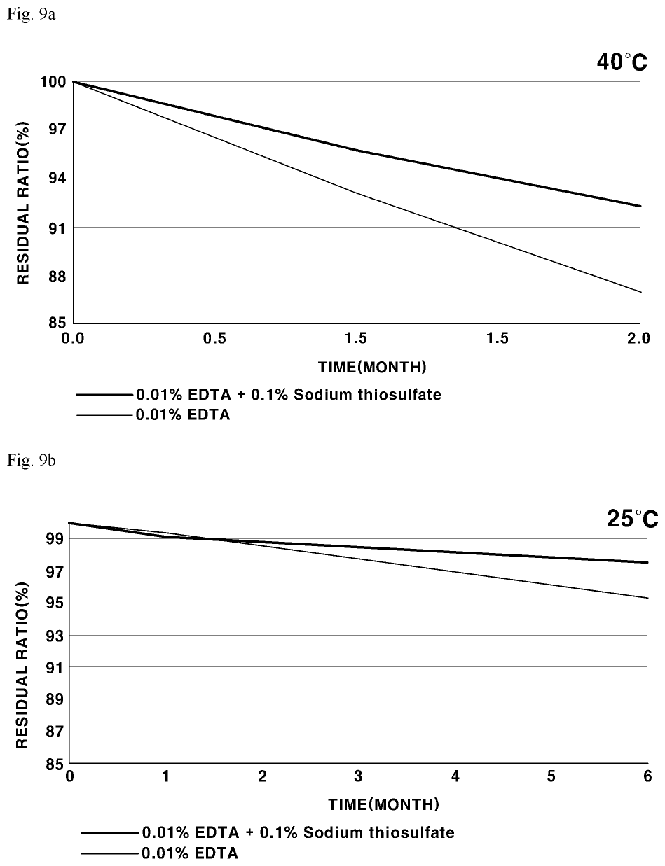

The results are shown in Table 12 and FIG. 7. The thermal stability of EW-7197 in the tyloxapol formulations decreased significantly by addition of BHT. Other stabilizing agents did not affect the thermal stability of EW-7197. When the thermal stability of EW-7197 in the formulation that does not contain stabilizing agent (F02) and the formulation that contains EDTA (F03) using both glass ampules and PE ophthalmic bottles was evaluated, the thermal stability was not improved by EDTA in these containers. However, although no difference was seen in the residual ratio of EW-7197, it was found that the production of degradation products was restrained by addition of EDTA. The HPLC chromatograms for the formulation that does not contain stabilizing agent (F02) and the formulation that contains EDTA (F03) are shown in FIG. 8. Then, the thermal stability of EW-7197 in the tyloxapol formulations that contain EDTA and sodium thiosulfate was evaluated using PE ophthalmic bottles as containers. The results are shown in Table 13 and FIG. 9. Although both of the formulations F120 and F125 contain EDTA, the thermal stability of EW-7197 was improved significantly in the formulation F125 which contains both EDTA and sodium thiosulfate. The residual ratio of EW-7197 in the formulation that contains sodium thiosulfate (F125) was 97.5% when stored for 6 months at 25° C., which was higher than 95.3%, the residual ratio of EW-7197 in the formulation that did not contain sodium thiosulfate (F120). Based on the above, sodium thiosulfate is effective for improving the thermal stability of EW-7197, and an estimated shelf-life of the formulation that contains sodium thiosulfate (F125) is expected to be 1.5 to 2 years at room temperature.

TABLE 12
Thermal stability with various stabilizing agents (40° C.)

| | | | | | | Residual ratio (%) | | | |
|---|---|---|---|---|---|---|---|---|---|
| Formulation | Stabilizing agent | | Storage | | EW-7197 | | 40° C. | 40° C. | 40° C. |
| number | Conc. (%) | Name | container | pH | Conc. (%) | Initial | 2 weeks | 1 month | 2 months |
| F02 | — | — | GA | 7.5 | 0.01 | 100.0 | 94.5 | 90.3 | 85.1 |
| | | | PE | 7.5 | 0.01 | 100.0 | 96.9 | 92.4 | 86.6 |
| F03 | 0.01 | EDTA | GA | 7.5 | 0.01 | 100.0 | 94.3 | 91.2 | 84.7 |
| | | | PE | 7.5 | 0.01 | 100.0 | 97.8 | 92.7 | 88.0 |
| F12 | 0.005 | BHT | GA | 7.5 | 0.01 | 100.0 | 88.8 | 80.1 | 65.4 |
| F05 | 0.1 | Mannitol | GA | 7.5 | 0.01 | 100.0 | 93.8 | 91.2 | 84.2 |
| F09 | 4 | PEG4000 | GA | 7.5 | 0.01 | 100.0 | 92.3 | 90.6 | 83.5 |
| F10 | 2 | Niacinamide | GA | 7.5 | 0.01 | 100.0 | 93.0 | 89.7 | 82.4 |
| F11 | 0.02 | HPMC 60SH-4000 | GA | 7.5 | 0.01 | 100.0 | 94.1 | 91.5 | 84.8 |

TABLE 13
Thermal stability of formulations with sodium thiosulfate (25° C. and 40° C.)

| | | | | | | Residual ratio (%) | | | | |
|---|---|---|---|---|---|---|---|---|---|---|
| Formulation | Stabilizing agent | | Storage | | EW-7197 | | 25° C. | 25° C. | 40° C. | 40° C. |
| number | Conc. (%) | Name | container | pH | Conc. (%) | Initial | 1 month | 6 months | 1 month | 2 months |
| F120 | 0.01 | EDTA | PE | 7.5 | 0.015 | 100.0 | 99.3 | 95.3 | 93.1 | 87.0 |
| F125 | 0.01 | EDTA | PE | 7.5 | 0.015 | 100.0 | 99.1 | 97.5 | 95.8 | 92.3 |
| | 0.1 | Sodium thiosulfate | | | | | | | | |

### Example 7. Thermal Stability of EW-7197 in Formulations with Various Buffers and pH Values

The thermal stability of EW-7197 in the tyloxapol formulations (containing niacinamide and PEG 4000) prepared by adding various buffers such as boric acid plus sodium borate, trometamol, boric acid plus trometamol, and sodium citrate was evaluated using PE ophthalmic bottles as containers as shown in Table 14. After the preparation of each formulation, the formulations were filtered. The filters used in the operation are shown in Table 2. The filtrate was filled into PE bottles, and stored in a thermostatic chamber (40° C.).

TABLE 14
Thermal stability test formulations with buffers and at various pH values

| Ingredient (g/100 mL)^{*6} | F46 | F92 | F62 | F94 | F96 | F88 |
|---|---|---|---|---|---|---|
| EW-7197 | 0.015 | 0.015 | 0.015 | 0.015 | 0.015 | 0.015 |
| Boric acid | 0.9 | — | — | 0.8 | 0.8 | — |
| Sodium borate (Bolax) | 0.1 | — | — | — | — | — |
| Trometamol | — | 1 | 1 | 0.8 | 0.8 | — |
| Trisodium citrate dihydrate | — | — | — | — | — | 0.2 |
| Tyloxapol | 1 | 1 | 1 | 1 | 1 | 1 |
| Disodium edetate dihydrate | 0.01 | 0.01 | 0.01 | 0.01 | 0.01 | 0.01 |
| (EDTA) | | | | | | |
| Polyethylene glycol 4000 | 4 | 4 | 4 | 4 | 4 | 4 |
| (PEG4000) | | | | | | |
| Niacinamide | 2 | 2 | 2 | 2 | 2 | 2 |
| Sodium hydrate | q.s. | q.s. | q.s. | q.s. | q.s. | q.s. |
| Hydrochloric acid | q.s. | q.s. | q.s. | q.s. | q.s. | q.s. |
| pH | 7.5 | 7.5 | 8.0 | 7.5 | 8.0 | 6.5 |

^{*6}Each formulation was prepared at the density of 1 g/mL.

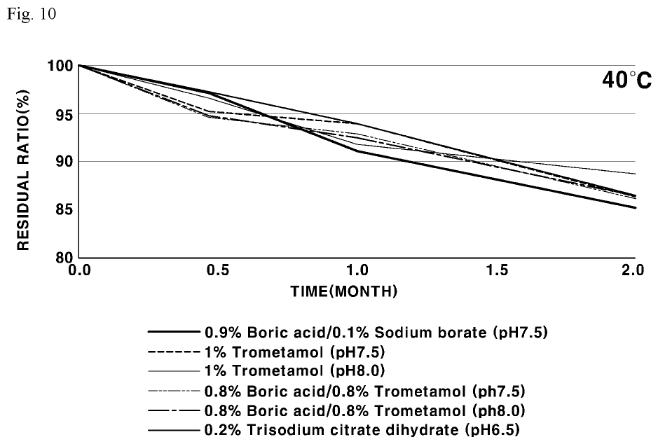

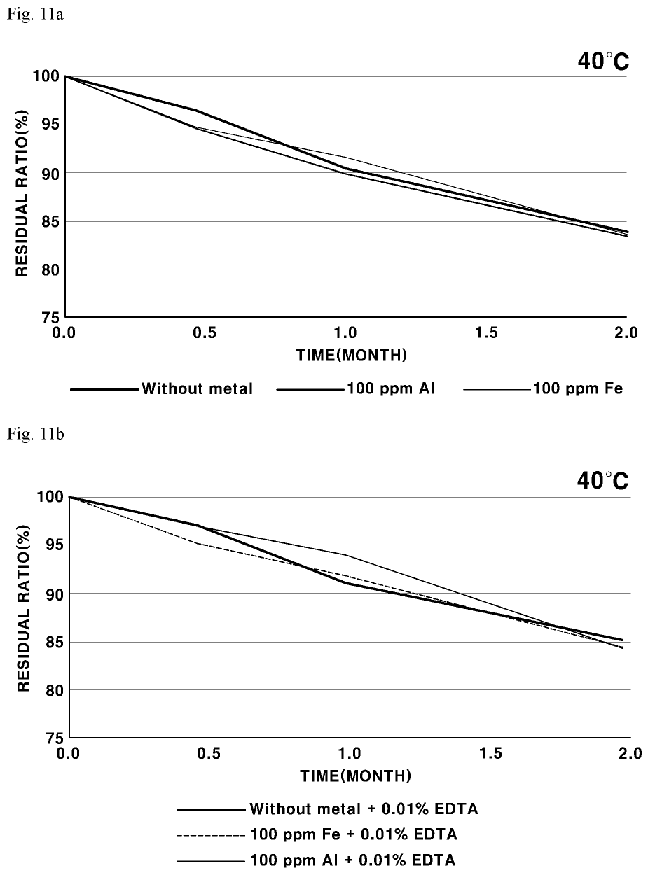

The results are shown in Table 15 and FIG. 10. The thermal stability of EW-7197 was higher in the formulations with trometamol, with boric acid, trometamol, and sodium citrate, compared to that in the formulation with boric acid plus sodium borate at pH7.5 (F46). In addition, in comparison of the formulation with trometamol at pH7.5 (F92) and the formulation with trometamol at pH8.0 (F62), the thermal stability of EW-7197 was higher in the formulation F62. The residual ratio of EW-7197 in the formulation F62 was 88.7% after stored for 2 months at 40° C. Based on the above, it was found that the most suitable buffer was trometamol for the formulation development of EW-7197 from the view point of thermal stability. Considering the expected preservation effect of boric acid from the view point of preservation of EW-7197, boric acid was included in the following experiment.

TABLE 15
Thermal stability in various buffers (40° C.)

| | | | | | | Residual ratio (%) | | | |
|---|---|---|---|---|---|---|---|---|---|
| Formulation | Buffering agent | | Storage | | EW-7197 | | 40° C. | 40° C. | 40° C. |
| number | Conc. (%) | Name | container | pH | Conc. (%) | Initial | 2 weeks | 1 month | 2 months |
| F46 | 0.9 | Boric acid | PE | 7.5 | 0.015 | 100.0 | 97.1 | 91.1 | 85.2 |
| | 0.1 | Sodium borate | | | | | | | |
| F92 | 1 | Trometamol | PE | 7.5 | 0.015 | 100.0 | 95.2 | 93.9 | 86.3 |
| F62 | 1 | Trometamol | PE | 8.0 | 0.015 | 100.0 | 96.6 | 91.8 | 88.7 |
| F94 | 0.8 | Boric acid | PE | 7.5 | 0.015 | 100.0 | 94.6 | 92.8 | 86.2 |
| | 0.8 | Trometamol | | | | | | | |
| F96 | 0.8 | Boric acid | PE | 8.0 | 0.015 | 100.0 | 94.8 | 92.5 | 86.5 |
| | 0.8 | Trometamol | | | | | | | |
| F88 | 0.2 | Trisodium citrate | PE | 6.5 | 0.015 | 100.0 | 97.2 | 93.9 | 86.5 |
| | | dihydrate | | | | | | | |

### Example 8. Thermal Stability of EW-7197 in Formulations with Metal Ion

Taking into account of the effect of trace metal, derived from manufacturing equipment in a plant or derived from excipients, the thermal stability of EW-7197 was evaluated in the formulations shown in Table 16 that contain aluminum or iron. After the preparation of each formulation, the formulation was filtered. The filters used in the operation are shown in Table 2. The filtrate was filled into a PE bottle, and stored in a thermostatic chamber (40° C.).

TABLE 16
Thermal stability test formulations with metal ion

| Ingredient (g/100 mL)^{*7} | F45 | F46 | 101 | 102 | 104 | 105 |
|---|---|---|---|---|---|---|
| EW-7197 | 0.015 | 0.015 | 0.015 | 0.015 | 0.015 | 0.015 |
| Boric acid | 0.9 | 0.9 | 0.9 | 0.9 | 0.9 | 0.9 |
| Sodium borate (Bolax) | 0.1 | 0.1 | 0.1 | 0.1 | 0.1 | 0.1 |
| Tyloxapol | 1 | 1 | 1 | 1 | 1 | 1 |
| Disodium edetate dihydrate | — | 0.01 | — | 0.01 | — | 0.01 |
| (EDTA) | | | | | | |
| Polyethylene glycol 4000 | 4 | 4 | 4 | 4 | 4 | 4 |
| (PEG4000) | | | | | | |
| Niacinamide | 2 | 2 | 2 | 2 | 2 | 2 |
| Aluminum^{*8} | — | — | 0.01 | 0.01 | — | — |
| Iron^{*8} | — | — | — | — | 0.01 | 0.01 |
| Sodium hydrate | q.s. | q.s. | q.s. | q.s. | q.s. | q.s. |
| Hydrochloric acid | q.s. | q.s. | q.s. | q.s. | q.s. | q.s. |
| pH | 7.5 | 7.5 | 7.5 | 7.5 | 7.5 | 7.5 |

^{*7}Each formulation was prepared at the density of 1 g/mL.
^{*8}Aluminum chloride and iron (II) chloride tetrahydrate were added to prepare the specified concentrations of aluminum and iron, respectively.

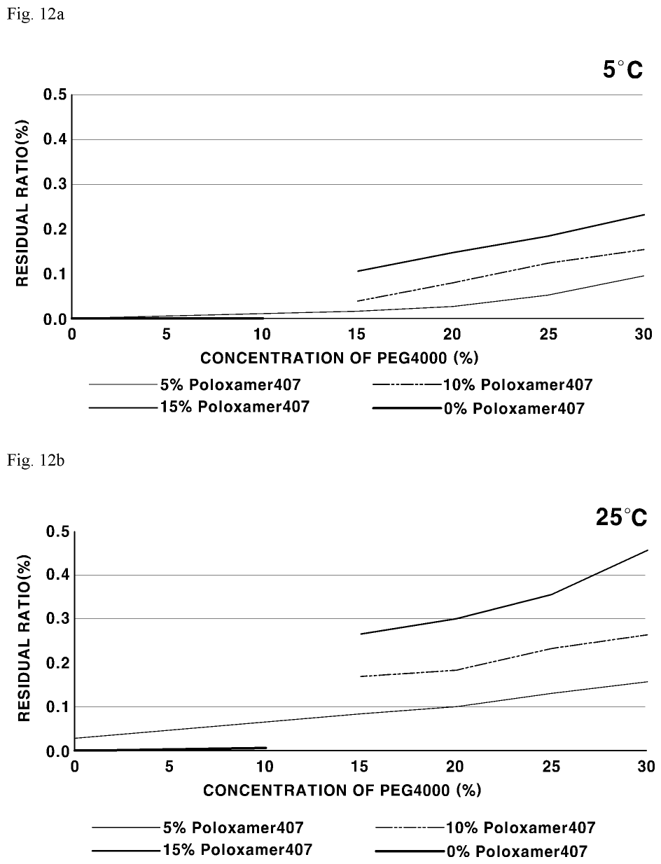

In this study, the final concentration of aluminum ions or iron ions was 100 ppm, and the PE ophthalmic bottles were used as containers. The results are shown in Table 17 and FIG. 11. Regardless of whether EDTA was added or not, neither aluminum ions nor iron ions affected the thermal stability of EW-7197. Based on the above, the trace metal derived from manufacturing equipment in a plant or derived from excipients does not affect the thermal stability of EW-7197.

TABLE 17
Effect of metal ion on thermal stability (40° C.)

| | | | | | | Residual ratio (%) | | | |
|---|---|---|---|---|---|---|---|---|---|
| Formulation | | Metal ion | EDTA | | EW-7197 | | 40° C. | 40° C. | 40° C. |
| number | Surfactant | Conc. (%) | Conc. (%) | pH | Conc. (%) | Initial | 2 weeks | 1 month | 2 months |
| F45 | 1% Tyloxapol | — | — | 7.5 | 0.015 | 100.0 | 96.5 | 90.5 | 83.9 |
| 101 | 1% Tyloxapol | 100 ppm Al | — | 7.5 | 0.015 | 100.0 | 94.6 | 89.9 | 83.5 |
| 104 | 1% Tyloxapol | 100 ppm Fe | — | 7.5 | 0.015 | 100.0 | 94.8 | 91.6 | 83.6 |
| F46 | 1% Tyloxapol | — | 0.01 | 7.5 | 0.015 | 100.0 | 97.1 | 91.1 | 85.2 |
| 102 | 1% Tyloxapol | 100 ppm Al | 0.01 | 7.5 | 0.015 | 100.0 | 95.2 | 91.9 | 84.5 |
| 105 | 1% Tyloxapol | 100 ppm Fe | 0.01 | 7.5 | 0.015 | 100.0 | 97.6 | 94.0 | 84.4 |

### Example 9-12 Illustrates Formulation Development of PEG/Poloxamer Formulation

Since 0.1% EW-7197 ophthalmic solution was not able to be formulated with ingredients of general ophthalmic solutions, the formulation development with the PEG/poloxamer formulations was further investigated.

### Example 9. Solubility of EW-7197 in the PEG/Poloxamer Formulations

The solubility of EW-7197 was evaluated in the PEG/poloxamer formulations at various concentrations as shown in Table 18. Each formulation was prepared at 30 mL scale, and after adding EW-7197, the formulations were stirred at 5° C. and 25° C. After confirming that the formulation was suspended at each temperature, the suspension was filtered. The EW-7197 content of the filtrate was measured by HPLC. The filters used in the operation are shown in Table 2.

TABLE 18
PEG/poloxamer formulations at various concentrations for solubility

| Ingredients (g/100 mL) | K01 | K02 | K03 | K04 | K05 | K06 | K07 | K08 | K09 | K10 | K11 | K12 |
|---|---|---|---|---|---|---|---|---|---|---|---|---|
| Boric acid | 0.9 | 0.9 | 9.0 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 |
| Poloxamer 407 | 5 | 5 | 5 | 5 | 10 | 10 | 10 | 10 | 15 | 15 | 15 | 15 |
| Polyethylene glycol 4000 | 15 | 20 | 25 | 30 | 15 | 20 | 25 | 30 | 15 | 20 | 25 | 30 |
| (PEG4000) | | | | | | | | | | | | |
| Disodium edetate | 0.01 | 0.01 | 0.01 | 0.01 | 0.01 | 0.01 | 0.01 | 0.01 | 0.01 | 0.01 | 0.01 | 0.01 |
| dihydrate (EDTA) | | | | | | | | | | | | |
| Sodium hydrate | q.s. | q.s. | q.s. | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 | 7.5 | 7.5 | 7.5 |

The results on solubility are shown in Table 19 and FIG. 12. The solubility of EW-7197 increased depending on the concentration of poloxamer 407. When PEG4000 was added at not less than 20%, the solubility of EW-7197 increased depending on the concentration of PEG4000 at every poloxamer 407 concentration. In the formulations K07, K08, K09, K10, K11, and K12, not less than 0.1% of EW-7197 was dissolved at 5° C. Although poloxamer 407 is an excipient with high safety profile, the concentration of poloxamer 407 should be at the lowest level possible since it is a surfactant. Therefore, a favorable concentration of poloxamer 407 is to be 10%.

TABLE 19
Solubility of EW-7197 in PEG/poloxamer formulation
(5° C. and 25° C.)

| | | | | 5° C. | 25° C. |
|---|---|---|---|---|---|
| | | Poloxamer | | EW-7197 | EW-7197 |
| Formulation | PEG4000 | 407 Conc. | | Solubility | Solubility |
| number | Conc. (%) | (%) | pH | (%) | (%) |
| K01 | 15 | 5 | 7.5 | 0.0182068 | 0.0853844 |
| K02 | 20 | 5 | 7.5 | 0.0274570 | 0.0998315 |
| K03 | 25 | 5 | 7.5 | 0.0539166 | 0.1318997 |
| K04 | 30 | 5 | 7.5 | 0.0947156 | 0.1583350 |
| K05 | 15 | 10 | 7.5 | 0.0392543 | 0.1709374 |
| K06 | 20 | 10 | 7.5 | 0.0805424 | 0.1851506 |
| K07 | 25 | 10 | 7.5 | 0.1246044 | 0.2341502 |
| K08 | 30 | 10 | 7.5 | 0.1540096 | 0.2654020 |
| K09 | 15 | 15 | 7.5 | 0.1058383 | 0.2670500 |
| K10 | 20 | 15 | 7.5 | 0.1474700 | 0.3018637 |
| K11 | 25 | 15 | 7.5 | 0.1840174 | 0.3569663 |
| K12 | 30 | 15 | 7.5 | 0.2311642 | 0.4572919 |

### Example 10. Thermal Stability of EW-7197 in PEG/Poloxamer Formulations

The thermal stability of EW-7197 was evaluated in the PEG/poloxamer formulations at various concentrations, in which EW-7197 was dissolved at 0.003%, 0.01%, 0.03%, and 0.1%, as shown in Table 21. After the preparation of each formulation, the formulations were filtered. The filters used in the operation are shown in Table 20. The filtrate was filled into PE bottles, and stored in a thermostatic chamber (40° C.).

TABLE 20
Filters used for filtration

| | Filter | Pore | Filter | Filtration |
|---|---|---|---|---|
| Filter name | material | size | diameter | area |
| Millipore Sterivex filter | PVDP | 0.45 μm | — | 10 cm² |
| unit SVHV010RS | | | | |
| Whatman GE healtheare FP 30 | — | 1.2 μm | 30 μm | 5.7 cm² |
| CA-S 1.2 μm | | | | |
| Whatman GE healtheare FP 30 | — | 5 μm | 30 μm | 5.7 cm² |
| CN-S 5 μm | | | | |

TABLE 21
Formulations with various concentrations of PEG/poloxamer formulations for thermal stability testing

| Ingredient (g/100 mL) | N01 | N02 | N03 | N04 | N05 | N06 | N07 | N08 | N09 | N10 | N11 | N12 | N13 | N14 |
|---|---|---|---|---|---|---|---|---|---|---|---|---|---|---|
| EW-7197 | 0.003 | 0.003 | 0.003 | 0.003 | 0.01 | 0.01 | 0.01 | 0.01 | 0.03 | 0.03 | 0.03 | 0.03 | 0.1 | 0.1 |
| Boric acid | 0.9 | 0.9 | 0.9 | 0.9 | 0.9 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 | 0.1 | 0.1 |
| Poloxamer 407 | 5 | 10 | 15 | 10 | 5 | 10 | 15 | 10 | 5 | 10 | 15 | 10 | 10 | 15 |
| Polyethylene glycol 4000 | 10 | 5 | 0 | 25 | 15 | 10 | 5 | 25 | 25 | 15 | 10 | 25 | 25 | 20 |
| (PEG4000) | | | | | | | | | | | | | | |
| Sodium hydrate | q.s. | q.s. | q.s. | q.s. | q.s. | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 | 7.5 | 7.5 | 7.5 | 7.5 | 7.5 |

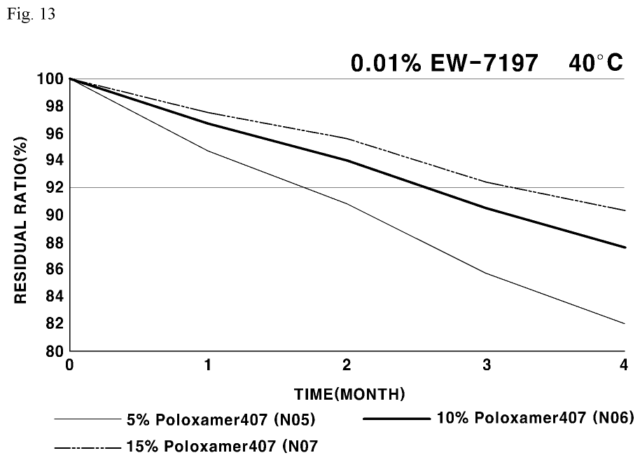

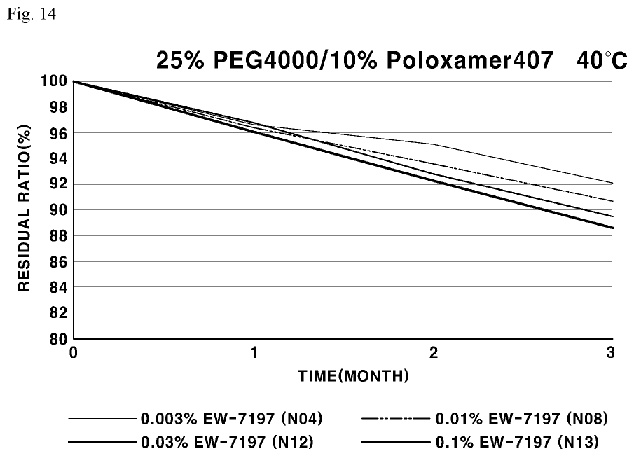

The results are shown in Table 22. When the concentration of EW-7197 was the same, the thermal stability of EW-7197 was higher in the formulation with higher concentration of poloxamer 407 as shown the results of the formulations that contain poloxamer 407 at 5% (N05), 10% (N06), and 15% (N07) in FIG. 13. In addition, the evaluation results at various concentrations of EW-7197 is described in FIG. 14. When setting the concentration of PEG4000 at 25% and that of poloxamer 407 at 10%, and comparing the thermal stability in the formulations at 4 concentration levels of EW-7197 (0.004%, 0.01%, 0.03%, and 0.1%), the thermal stability decreased as the level of EW-7197 concentration increases. It seems that EW-7197 is stabilized when bound in micelles of poloxamer 407, while it is degraded outside of the micelles by the effects of water molecules, etc. The appearance of each formulation was clear and colorless with no foreign insoluble matters were detected at the start of storage, and no change was observed after the storage. Based on the results on the solubility and thermal stability of EW-7197, 25% PEG4000 and 10% poloxamer 407 was chosen for further investigation as they were capable of dissolving EW-7197 at not more than 0.1% and provided relatively high thermal stability to EW-7197.

TABLE 22
Thermal stability at various concentrations of PEG/poloxamer formulation (40° C.)

| | | | | | | Residual ratio (%) | | | | |
|---|---|---|---|---|---|---|---|---|---|---|
| Formulation | PEG4000 | Poloxamer 407 | Storage | | EW-7197 | | 40° C. | 40° C. | 40° C. | 40° C. |
| number | Conc. (%) | Conc. (%) | container | pH | Conc. (%) | Initial | 1 month | 2 months | 3 months | 4 months |
| N01 | 10 | 5 | PE | 7.5 | 0.003 | 100.0 | 96.1 | 92.3 | 88.8 | 85.1 |
| N02 | 5 | 10 | PE | 7.5 | 0.003 | 100.0 | 97.4 | 95.7 | 92.7 | 89.7 |
| N03 | 0 | 15 | PE | 7.5 | 0.003 | 100.0 | 97.7 | 94.0 | 94.7 | 92.0 |
| N04 | 25 | 10 | PE | 7.5 | 0.003 | 100.0 | 96. [symbol] | 95.1 | 92.1 | 89.5 |
| N05 | 15 | 5 | PE | 7.5 | 0.01 | 100.0 | 94.7 | 90.8 | 85.7 | 82.0 |
| N06 | 10 | 10 | PE | 7.5 | 0.01 | 100.0 | 96.7 | 94.0 | 90. [symbol] | 87.6 |
| N07 | 5 | 15 | PE | 7.5 | 0.01 | 100.0 | 97.5 | 95.6 | 92.4 | 96.3? |
| N08 | 25 | 10 | PE | 7.5 | 0.01 | 100.0 | 96.4 | 93.6 | 90.7 | 87.8 |
| N09 | 25 | 5 | PE | 7.5 | 0.03 | 100.0 | 94.4 | 89.8 | 84.7 | 80.7 |
| N10 | 15 | 10 | PE | 7.5 | 0.03 | 100.0 | 96.2 | 92.8 | 88.8 | 85.9 |
| N11 | 10 | 15 | PE | 7.5 | 0.03 | 100.0 | 97.3 | 94.9 | 90.7 | 88.5 |
| N12 | 25 | 10 | PE | 7.5 | 0.03 | 100.0 | 96.8 | 92.8 | 89.5 | 86.5 |
| N13 | 25 | 10 | PE | 7.5 | 0.1 | 100.0 | 96.1 | 92.3 | 88.6 | 85.5 |
| N14 | 20 | 15 | PE | 7.5 | 0.1 | 100.0 | 97.6 | 95.0 | 91.5 | 88.8 |

[symbol]

indicates data missing or illegible when filed

### Example 11. Thermal Stability of EW-7197 in PEG/Poloxamer Formulations Containing Various Stabilizing Agents

The thermal stability of EW-7197 was evaluated in the formulations that contain various stabilizing agents at pH7.5, 8.0 and 8.3 as shown in Table 23. After the preparation of each formulation, the formulations were filtered. The filters used in the operation are shown in Table 20. The filtrate was filled into PE bottles, and stored in a thermostatic chamber (40° C.).

TABLE 23
Thermal stability test formulations with various stabilizing agents

| Ingredients (g/100 mL) | N36 | N08 | N38 | N37 | N16 | N17 | N40 | N23 | N42 | N41 | N24 | N25 | N22 |
|---|---|---|---|---|---|---|---|---|---|---|---|---|---|
| EW-7197 | 0.01 | 0.01 | 0.01 | 0.01 | 0.01 | 0.01 | 0.01 | 0.01 | 0.01 | 0.01 | 0.01 | 0.01 | 0.01 |
| Boric acid | 0.9 | 0.9 | 0.9 | 0.9 | 0.9 | 0.9 | 0.9 | 0.9 | 0.9 | 0.9 | 0.9 | 0.9 | 0.9 |
| Sodium borate (Bolax) | 0.1 | 0.1 | 0.1 | 0.1 | 0.1 | 0.1 | 0.1 | 0.1 | 0.1 | 0.1 | 0.1 | 0.1 | 0.1 |
| Poloxamer 407 | 10 | 10 | 10 | 10 | 10 | 10 | 10 | 10 | 10 | 10 | 10 | 10 | 10 |
| Polyethylene glycol 4000 | 25 | 25 | 25 | 25 | 25 | 25 | 25 | 25 | 25 | 25 | 25 | 25 | 25 |
| (PEG4000) | | | | | | | | | | | | | |
| Disodium edetate dihydrate | — | 0.01 | — | — | 0.01 | 0.01 | — | 0.01 | — | — | 0.01 | 0.01 | 0.01 |
| (EDTA) | | | | | | | | | | | | | |
| Dibutyl hydroxyl toluene | — | — | 0.005 | — | 0.005 | — | — | — | 0.005 | — | 0.005 | — | — |
| (BHT) | | | | | | | | | | | | | |
| Sodium thiosulfate hydrate | — | — | — | 0.1 | — | 0.1 | — | — | — | 0.1 | — | 0.1 | — |
| Sodium hydrate | q.s. | q.s. | q.s. | q.s. | q.s. | q.s. | q.s. | q.s. | q.s. | q.s. | q.s. | q.s. | q.s. |
| Hydrochloric acid | q.s. | q.s. | q.s. | q.s. | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 | 7.5 | 7.5 | 7.5 | 7.5 |

The results are shown in Table 24. No difference in thermal stability was seen at various pH values in the formulations N36 (pH7.5), N40 (pH8.0), and N22 (pH8.3). Although the thermal stability of EW-7197 was not improved when adding EDTA to the tyloxapol formulations, the thermal stability was improved in both of the PEG/poloxamer formulations N08 (pH7.5) and N23 (pH8.0) that contain EDTA. In addition, the thermal stability was also improved in the formulations N37 (pH7.5) and N41 (pH8.0) that contain sodium thiosulfate. The thermal stability was improved slightly more in the formulations N17 (pH7.5) and N25 (pH8.0) that contain both EDTA and sodium thiosulfate, when compared to the formulations that contain one type of stabilizing agent. The residual ratio of EW-7197 in the formulation N25 was 94.7% after stored for 2 months at 40° C. Meanwhile, for the formulations N38 (pH7.5) and N42 (pH8.0) that contain BHT, no change was observed in thermal stability in the formulation N38, and the thermal stability was improved slightly in the formulation N42. The appearance of each formulation was clear and colorless with no foreign insoluble matters detected at the start of storage, and no change was observed after the storage. Based on the above, we found that the thermal stability of EW-7197 was improved in the PEG/poloxamer formulations containing EDTA or sodium thiosulfate, and the thermal stability was improved further by adding both EDTA and sodium thiosulfate to the formulation. A current estimated shelf-life of the formulation N25 (pH8.0) is 1.5 to 2 years at room temperature.

TABLE 24
Thermal stability with various stabilizing agents (40° C.)

| | | | | | | Residual ratio (%) | | | | |
|---|---|---|---|---|---|---|---|---|---|---|
| Formulation | Stabilizing agent | | Storage | | EW-7197 | | 40° C. | 40° C. | 40° C. | 40° C. |
| number | Conc. (%) | Name | container | pH | Conc. (%) | Initial | 1 month | 2 months | 3 months | 4 months |
| N36 | — | — | PE | 7.5 | 0.01 | 100.0 | 94.4 | — | — | — |
| N08 | 0.01 | EDTA | PE | 7.5 | 0.01 | 100.0 | 96.4 | 93.8 | 90.7 | 87.8 |
| N38 | 0.005 | BHT | PE | 7.5 | 0.01 | 100.0 | 94.6 | — | — | — |
| N37 | 0.1 | Sodium thiosulfate | PE | 7.5 | 0.01 | 100.0 | 97.1 | — | — | — |
| N16 | 0.01 | EDTA | PE | 7.5 | 0.01 | 100.0 | 96.9 | 92.7 | — | — |
| | 0.005 | BHT | | | | | | | | |
| N17 | 0.01 | EDTA | PE | 7.5 | 0.01 | 100.0 | 97.6 | 94.3 | — | — |
| | 0.1 | Sodium thiosulfate | | | | | | | | |
| N40 | — | — | PE | 8.0 | 0.01 | 100.0 | 93.3 | — | — | — |
| N23 | 0.01 | EDTA | PE | 8.0 | 0.01 | 100.0 | 96.3 | 92.4 | — | — |
| N42 | 0.005 | BHT | PE | 8.0 | 0.01 | 100.0 | 95. [symbol] | — | — | — |
| N41 | 0.1 | Sodium thiosulfate | PE | 8.0 | 0.01 | 100.0 | 96.3 | — | — | — |
| N24 | 0.01 | EDTA | PE | 8.0 | 0.01 | 100.0 | 96.7 | 93.0 | — | — |
| | 0.005 | BHT | | | | | | | | |
| N25 | 0.01 | EDTA | PE | 8.0 | 0.01 | 100.0 | 97.0 | 94. [symbol] | — | — |
| | 0.1 | Sodium thiosulfate | | | | | | | | |
| N22 | 0.01 | EDTA | PE | 8.3 | 0.01 | 100.0 | 96.8? | 92.8 | — | — |

[symbol]

indicates data missing or illegible when filed

### Example 12. Thermal Stability of EW-7197 in PEG/Poloxamer Formulations Containing Various Viscous Agents

The thermal stability of EW-7197 was evaluated in the formulations that contain various viscous agents as shown in Table 25. After the preparation of each formulation, the formulation was filtered. The filters used in the operation are shown in Table 20. The filtrate was filled into a PE bottle, and stored in a thermostat bath (40° C.).

TABLE 25
Thermal stability test formulations with various viscous agents

| Ingredient (g/100 mL) | N29 | N30 | N31 | N32 |
|---|---|---|---|---|
| EW-7197 | 0.001 | 0.001 | 0.001 | 0.001 |
| Boric acid | 0.9 | 0.9 | 0.9 | 0.9 |
| Sodium borate (Bolax) | 0.1 | 0.1 | 0.1 | 0.1 |
| Poloxamer 407 | 10 | 10 | 10 | 10 |
| Polyethylene glycol 4000 | 25 | 25 | 25 | 25 |
| (PEG4000) | | | | |
| Disodium edetate dihydrate | 0.01 | 0.01 | 0.01 | 0.01 |
| (EDTA) | | | | |
| Xantan gum | 0.01 | 0.01 | — | — |
| Carmellose sodim CMC) | — | — | 0.1 | 0.05 |
| Sodium hydrate | q.s. | q.s. | q.s. | q.s. |
| Hydrochloric acid | q.s. | q.s. | q.s. | q.s. |
| pH | 7.5 | 7.5 | 7.5 | 7.5 |

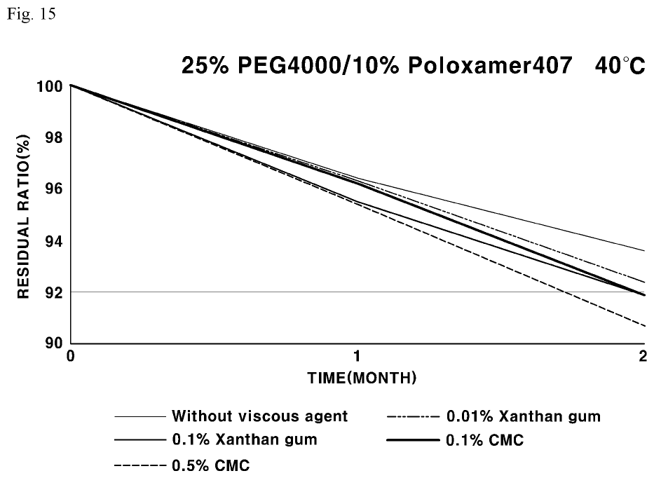

The results are shown in Table 26 and FIG. 15. The thermal stability decreased in the formulations that contain xanthan gum or carmellose sodium, when compared to the formulations that did not contain these viscous agents. In addition, the thermal stability decreased depending on the concentration levels of these agents. It seemed that the formulation became unstable because the distribution ratio of EW-7197 out of poloxamer 407 micelle increased by adding water-soluble polymers such as xanthan gum and carmellose sodium. The appearance of each formulation was clear and colorless with no foreign insoluble matters detected at the start of storage, and no change was observed after the storage. Based on the above, when adding water-soluble polymers for the purpose of viscous, it is considered better to add them at the lowest amount possible.

TABLE 26
Thermal stability with various viscous agents (40° C.)

| | | | | | | Residual ratios (%) | | | | |
|---|---|---|---|---|---|---|---|---|---|---|
| Formulation | Viscous agent | | Storage | | EW-7197 | | 40° C. | 40° C. | 40° C. | 40° C. |
| number | Conc. (%) | Name | container | pH | Conc. (%) | Initial | 1 month | 2 months | 3 months | 4 months |
| N08 | — | — | PE | 7.5 | 0.01 | 100.0 | 96.4 | 93.6 | 90.7 | 87.8 |
| N29 | 0.01 | Xanthan gum | PE | 7.5 | 0.01 | 100.0 | 96.3 | 92.4 | — | — |
| N30 | 0.1 | Xanthan gum | PE | 7.5 | 0.01 | 100.0 | 95.5 | 91.9 | — | — |
| N31 | 0.1 | CMC | PE | 7.5 | 0.01 | 100.0 | 96.2 | 91.9 | — | — |
| N32 | 0.5 | CMC | PE | 7.5 | 0.01 | 100.0 | [symbol] | 90.7 | — | — |

[symbol]

indicates data missing or illegible when filed

In conclusion, the solubility of EW-7197 in the PEG/poloxamer formulations was very high. The solubility of EW-7197 was about 0.125% (5° C.) in formulations containing 25% PEG4000 and 10% poloxamer 407. The thermal stability of EW-7197 in the PEG/poloxamer formulation was greatly affected by the concentration of poloxamer 407. Sodium thiosulfate was effective to improve the stability of EW-7197 in the PEG/poloxamer formulation, as well as in the tyloxapol formulations. Meanwhile, contrarily to its small effect in the tyloxapol formulations, EDTA was also effective to improve the stability of EW-7197 in the PEG/poloxamer formulation. It seemed that viscous agents increased the distribution ratio of EW-7197 out of poloxamer 407 micelles, and the thermal stability of EW-7197 decreased when viscous agents were added. The residual ratio of EW-7197 in the formulation prepared by adding sodium thiosulfate and EDTA to 25% PEG4000/10% poloxamer 407 was 94.7% after stored for 2 months at 40° C. A current estimated shelf-life of the formulation is 1.5 to 2 years at room temperature.

The effects of the present invention are not limited to those mentioned above. It should be understood that the effects of the present invention include all effects that can be inferred from the description of the present invention.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A composition for treating or preventing a disease state mediated by transforming growth factor-β (TFG-β) type I receptor (ALK5) in human, comprising an effective amount of EW-7197 (2-fluoro-N-((5-(6-methylpyridin-2-yl)-4-([1,2,4]triazolo[1,5-a]pyridin-6-yl)-1H-imidazol-2-yl)methyl)aniline) or a pharmaceutically acceptable salt thereof, or a solvate thereof.

2. The composition of claim 1, wherein the composition is for reducing the accumulation of excess extracellular matrix (ECM) in human by inhibiting the TFG-β signaling pathway, for example, inhibiting the phosphorylation of Smad2 or Smad3 by ALK5.

3. The composition of claim 1, wherein a disease state mediated by ALK5 or accumulating excess ECM by inhibiting the TFG-β signaling pathway is glaucoma, glaucoma filtration surgery bleb failure, intraocular pressure, cataract, posterior capsule opacification (PCO; secondary cataract), corneal haze, wet age-related macular degeneration (AMD), proliferative vitreoretinopathy (PVR), proliferative diabetic retinopathy (PDR), Fuchs' endothelial corneal dystrophy (FECD), congenital ectopia lentis (CEL), postsurgical peritoneal adhesions, postsurgical anastomotic strictures, hypertrophic scar, or keloid.

4. The composition of claim 1, wherein EW-7197 or a pharmaceutically acceptable salt thereof, or a solvate thereof is in the composition at a concentration of about 0.001% w/v to about 0.5% w/v.

5. The composition of claim 1, wherein EW-7197 or a pharmaceutically acceptable salt thereof, or a solvate thereof is in the composition at a concentration of about 0.01% w/v to about 0.2% w/v.

6. The composition of claim 1, wherein said composition further comprises an ophthalmologically acceptable solvent, diluting agent, liquid vehicle, preservative, stabilizer, solubilizer, viscosity enhancer, penetration enhancer, tonicity agent, gelling agent, buffering agent, wetting agent, or antioxidant.

7. The composition of claim 4, wherein said composition further comprises an ophthalmologically acceptable solvent, diluting agent, liquid vehicle, preservative, stabilizer, solubilizer, viscosity enhancer, penetration enhancer, tonicity agent, gelling agent, buffering agent, wetting agent, or antioxidant.

8. The composition of claim 5, wherein said composition further comprises an ophthalmologically acceptable solvent, diluting agent, liquid vehicle, preservative, stabilizer, solubilizer, viscosity enhancer, penetration enhancer, tonicity agent, gelling agent, buffering agent, wetting agent, or antioxidant.

9. The composition of claim 6, wherein said solubilizer is tyloxapol, polysorbate 80, PEG-40 stearate (MYS-40), PEG-60 hydrogenated castor oil (HCO-60), poloxamer, polyethylene glycol (PEG), PEG/tyloxapol, or PEG/poloxamer.

10. The composition of claim 6, wherein preferred solubilizer is PEG/poloxamer.

11. The composition of claim 9, wherein preferred poloxamer is poloxamer 188 or poloxamer 407, and preferred PEG is PEG4000.

12. The composition of claim 1, which is an eye drop.
